SEQUENCE LISTING (38 sequences)

SEQ ID NO: 1 - protein, 290 aa, synthetic construct
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 2 - protein, 290 aa, synthetic construct
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 3 - protein, 221 aa, synthetic construct [sequence omitted]

SEQ ID NO: 4 - protein, 115 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAP

SEQ ID NO: 5 - protein, 109 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRIT

SEQ ID NO: 6 - protein, 107 aa, synthetic construct
VTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRIT

SEQ ID NO: 7 - protein, 106 aa, synthetic construct
YNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERT

SEQ ID NO: 8 - protein, 105 aa, synthetic construct
YNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 9 - protein, 107 aa, synthetic construct
ITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRIT

SEQ ID NO: 10 - protein, 7 aa, synthetic construct
GGGSGGS

SEQ ID NO: 11 - protein, 223 aa, synthetic construct
THTCPPCPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 451 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTGGGSGGSTHTCPPCPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 345 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPGGGSGGSTHTCPPCPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 30 aa, synthetic construct
RNTGRGGEEKKKEKEKEEQEERETKTPECP

SEQ ID NO: 15 - protein, 466 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 8 aa, synthetic construct
THTCPPCP

SEQ ID NO: 17 - protein, 20 aa, synthetic construct
AAGSGGGGGSGGGGSGGGGS

SEQ ID NO: 18 - protein, 5 aa, synthetic construct
GGSGG

SEQ ID NO: 19 - protein, 9 aa, synthetic construct
GGSGGSGGS

SEQ ID NO: 20 - protein, 6 aa, synthetic construct
GGGSGG

SEQ ID NO: 21 - protein, 18 aa, synthetic construct
KESGSVSSEQLAQFRSLD

SEQ ID NO: 22 - protein, 14 aa, synthetic construct
EGKSSGSGSESKST

SEQ ID NO: 23 - protein, 12 aa, synthetic construct
GSAGSAAGSGEF

SEQ ID NO: 24 - protein, 12 aa, synthetic construct
CRRRRRREAEAC

SEQ ID NO: 25 - protein, 8 aa, synthetic construct
GGGGGGGG

SEQ ID NO: 26 - protein, 6 aa, synthetic construct
GGGGGG

SEQ ID NO: 27 - protein, 13 aa, synthetic construct
AEAAAKEAAAAKA

SEQ ID NO: 28 - protein, 5 aa, synthetic construct
PAPAP

SEQ ID NO: 29 - protein, 17 aa, synthetic construct
VSQTSKLTRAETVFPDV

SEQ ID NO: 30 - protein, 6 aa, synthetic construct
PLGLWA

SEQ ID NO: 31 - protein, 10 aa, synthetic construct
TRHRQPRGWE

SEQ ID NO: 32 - protein, 10 aa, synthetic construct
AGNRVRRSVG

SEQ ID NO: 33 - protein, 8 aa, synthetic construct
RRRRRRRR

SEQ ID NO: 34 - protein, 4 aa, synthetic construct
GFLG

SEQ ID NO: 35 - protein, 31 aa, synthetic construct
GSSGGSGSSGGSGGGDEADGSRGSQKAGVDE

SEQ ID NO: 36 - protein, 6 aa, synthetic construct
GSSGGS

SEQ ID NO: 37 - protein, 5 aa, synthetic construct
EAAAK

SEQ ID NO: 38 - protein, 46 aa, synthetic construct
AEAAAKEAAAKEAAAKEAAAKALEAEAAAKEAAAKEAAAKEAAAKA

(19) United States
(12) Patent Application Publication
(10) Pub. No.: US 2025/0382347 A1
(43) Pub. Date: Dec. 18, 2025

(54) Methods For Reducing Or Preventing Cerebral Edema After Stroke

(71) Applicant: The Johns Hopkins University, Baltimore, MD (US)

(72) Inventors: Jennifer Kim, Baltimore, MD (US); Christopher Jackson, Baltimore, MD (US); Michael Lim, Baltimore, MD (US)

(21) Appl. No.: 19/229,456

(22) Filed: Jun. 5, 2025

Related U.S. Application Data
(60) Provisional application No. 63/660,288, filed on Jun. 14, 2024.

(51) Int. Cl. C07K 14/705; A61K 9/00; A61K 38/00; A61K 39/00; A61P 25/28; C07K 16/28

(52) U.S. Cl. CPC C07K 14/70532; A61K 9/0019; A61P 25/28; C07K 16/2818; A61K 38/00; A61K 2039/542; C07K 2319/00

(57) ABSTRACT

Disclosed herein are methods of reducing or preventing cerebral edema in a subject after a stroke by administering systemically a therapeutically effective amount of a PD-1 agonist to the subject. Also disclosed herein are methods of treating neuroinflammation, improving gait, improving sensorimotor deficits, reducing the number of PD-1 positive monocytes in the brain, decreasing intracranial pressure, shifting the phenotype of monocytes in the brain from a classical inflammatory subtype to a non-classical subtype, and limiting or reducing secondary inflammatory injury, reducing a risk of a second or more stroke events in a subject after a stroke.

### CROSS REFERENCE TO RELATED APPLICATIONS

This application claims the benefit of the filing date of U.S. Provisional Application No. 63/660,288, filed on Jun. 14, 2024. The content of this earlier filed application is hereby incorporated by reference in its entirety.

### REFERENCE TO A SEQUENCE LISTING

The present application contains a Sequence Listing that is submitted concurrent with the filing of this application in XML format, containing the file name “36406_0039U2_SL.xml,” created on May 16, 2025, and having a size of 36,864 bytes. The Sequence Listing is hereby incorporated by reference pursuant into the present application in its entirety.

### BACKGROUND

Stroke can be fatal and is associated with long-term disability. Most strokes are ischemic, and large vessel occlusions (LVOs) account for 20-40% of acute ischemic strokes (K. Malhotra, et al, Front Neurol. 8, 651 (2017); and W. S. Smith, et al., Stroke. 40, 3834-3840 (2009)). LVOs are associated with larger infarct volumes and contribute to a disproportionately higher rate of post-stroke dependence and mortality (K. Malhotra, J. Gornbein, and J. L. Saver, Front Neurol. 8, 651 (2017)). Currently treatment focuses on restoring cerebral blood flow using intravenous thrombolytics or mechanical thrombectomy to minimize ischemic cell death. The care that follows is supportive. Thus, a need exists for better treatments after stroke.

### SUMMARY OF THE INVENTION

Disclosed herein are methods of reducing or preventing cerebral edema in a subject after a stroke, the methods comprising: administering systemically a therapeutically effective amount of a PD-1 agonist to the subject.

Disclosed herein are methods of treating neuroinflammation in a subject after a stroke, the methods comprising: administering systemically a therapeutically effective amount of a PD-1 agonist to the subject.

Disclosed herein are methods of improving gait in a subject after a stroke, the methods comprising: administering systemically a therapeutically effective amount of a PD-1 agonist to the subject.

Disclosed herein are methods of improving sensorimotor deficits in a subject after a stroke, the methods comprising: administering systemically a therapeutically effective amount of a PD-1 agonist to the subject.

Disclosed herein are methods of reducing the number of PD-1 positive monocytes in the brain of a subject after a stroke, the method comprising: administering systemically a therapeutically effective amount of a PD-1 agonist to the subject.

Disclosed herein are methods of decreasing intracranial pressure in a subject after a stroke, the methods comprising: administering systemically a therapeutically effective amount of a PD-1 agonist to the subject.

Disclosed herein are methods of shifting the phenotype of monocytes in the brain from a classical inflammatory subtype to a non-classical subtype in a subject after a stroke, the methods comprising: administering systemically a therapeutically effective amount of a PD-1 agonist to the subject.

Disclosed herein are methods of limiting or reducing secondary inflammatory injury in a subject after a stroke, the method comprising: administering systemically a therapeutically effective amount of a PD-1 agonist to the subject.

Disclosed herein are methods of reducing a risk of a second or more stroke events in a subject after a stroke, the method comprising: administering systemically a therapeutically effective amount of a PD-1 agonist to the subject.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-G show PD-1 is upregulated on circulating monocytes after stroke. FIG. 1A shows representative patient images of an acute right MCA occlusion on CTA, and associated MRI images demonstrating a large MCA territory infarct (ADC/DWI) with associated edema (T2). FIG. 1B depicts flow cytometric analysis showing frequencies of monocyte subsets in the blood during the acute period after MCA occlusion and reperfusion. FIG. 1C shows changes in PD-1 expression on blood monocytes after MCA occlusion and reperfusion. FIG. 1D shows PD-1 mean fluorescence intensity (MFI) on blood monocytes after MCA occlusion and reperfusion in a cohort of 14 patients. FIGS. 1E, IF show correlation analysis of maximum and average PD-1 MFI on circulating monocytes with edema-to-infarct ratio. FIG. 1G shows correlation analysis of maximum and average PD-1 MFI on monocyte subsets with edema-to infarct ratio.

FIGS. 2A-G show that PD-L1 attenuates brain injury and improves survival after MCAO. FIG. 2A shows the Kaplan-Meier curve. 30-day survival rate after MCAO was significantly increased by treatment with soluble PD-L1. P<0.0022, log-rank test; n=19 to 56 mice per experimental arm. FIGS. 2B, 2C show bar graphs that demonstrate that brain water content is increased at 48 hours after MCAO. Treatment decreases water content, though not back to baseline. P<0.0001 sham versus MCAO; p=0.0003 sham versus MCAO+PD-L1; p=0.0266 MCAO versus MCAO+PD-L1N=8-10 mice per experimental group. FIG. 2D show representative MRI images of wild type mice, 72 hours after MCAO. ADC and TRACE sequences show a right MCA territory infarct pattern. The T2-weighted sequence demonstrates brain edema (high signal intensity) within and surrounding the core infarct. FIG. 2E show a bar graph that demonstrates no significant change in infarct volume with treatment (p=0.3295). However, treatment significantly decreases the volume of edema surrounding the core infarct (FIG. 2F), p=0.0001, and volume of total edema per volume of core infarct (FIG. 2G), p<0.0001. n=14 to 15 per experimental group. Statistical analyses were performed by Mann-Whitney test. Error bars represent +/−SEM.

FIGS. 3A-G show that PD-L1 therapy improves short- and long-term functional outcomes. FIG. 3A depicts a bar chart that shows the 28-point neuroscores at week 1 for wild-type mice categorized by quartile (1st, 2nd, 3rd, 4th). Statistical analyses were performed by Chi-square and Fisher exact tests to evaluate the association between treatment with PD-L1 and having a high 28-point neuroscore (4th quartile). P=0.011, n=35 to 38 per experimental group. FIG. 3B shows a graphical representation of select gait parameters (FIGS. 3C, 3D) Bar graphs depict percent change in median when compared to sham mice. Displayed are all the variables that were significantly modified by stroke for the Digigait test at week 1 and week 3. FIG. 3E shows representative tracings of the mouse's trajectory in an open field. FIGS. 3C, 3D show bar graphs that depict percent change in median when compared to sham mice. Displayed are all the variables that were significantly modified by stroke for the Digigait test at week 1 (FIG. 3C) and week 3 (FIG. 3E), as well as the open field test at week 4 (FIG. 3F). Significant p<0.05 when comparing MCAO and sham after a Bonferroni correction for multiple comparisons. FIG. 3G shows a table that summarizes the number of variables that, with treatment, more closely resembled sham animals than untreated MCAO animals.

FIGS. 4A-I show brain infiltrating myeloid cells are mediators of the treatment effect. FIG. 4A shows representative flow cytometry plots to illustrate gating strategy for PD-1+ monocytes (CD11b+CD45hi). FIG. 4B shows MCAO results in significant infiltration of monocytes into the ischemic hemisphere (p=0.0159) at 48 hours after MCAO. Though the total numbers are not significantly affected by treatment, PD-L1 administration significantly decreases the frequency of PD-1+ monocytes (FIG. 4C) and overall expression as measured by mean fluorescence intensity (MFI) (FIG. 4D). Experiments run in duplicate with n≥4 mice per arm. FIG. 4E shows representative MRI images of global myeloid knockout (PD-1^{-/-}) mice and myeloid-specific knockout (PD-1^{f/fLysMcre}) mice, 72 hours after MCAO. ADC (low signal intensity) and TRACE (high signal intensity) sequences depict the core infarct, while the T2-weighted (high signal intensity) sequence demonstrates edema. FIG. 4F shows a bar graph that demonstrates no significant change in the volume of edema surrounding the core infarct or for the volume of total edema per volume of core infarct (FIG. 4G). N=8 per group for the global PD-1 knockout arms, and n=4 to 7 for the myeloid-specific knockout arms. Statistical analyses were performed by Mann-Whitney test. Error bars represent +/−SEM. FIGS. 4H-I shows a bar chart that depicts the 28-point neuroscores at week 1 for global and tissue-specific PD-1 knockout mice categorized by quartiles. Statistical analyses using Chi-square and Fisher exact tests demonstrated no significant association between PD-L1 treatment and having a high 28-point neuroscore (4^{th} quartile). N=25 to 27 per group for the global PD-1 knockout arms, and n=4 to 8 for the myeloid-specific PD-1 knockout arms.

FIGS. 5A-I show sPD-L1 after MCAO reprograms circulating monocytes to a restorative subtype with downregulation of inflammatory markers and increased metabolic reserve. FIG. 5A shows a UMAP of PD-L1 treated and untreated monocytes single cell RNA sequencing demonstrated the transcriptional heterogeneity of PD-L1 treated and untreated murine monocytes isolated at 48 hours after MCAO. FIG. 5B depicts a volcano plot showing 5,455 significantly differentially expressed genes (Benjamini-Hochberg adjusted p<0.05). Horizontal blue line indicates pmin, which is the smallest p-value represented in R (˜2.23 e-308). Points at the blue line indicate genes with p-value <pmin. FIG. 5C shows the top 15 positively (red) and negatively (blue) enriched gene sets from Gene Ontology: Biological Processes. FIG. 5D depicts a UMAP showing 11 transcriptional clusters derived from Louvain community detection. FIG. 5E shows a table demonstrating number of PD-L1 treated cells, total cells and proportion of treated cells in each cluster and p-value of differential proportion test between proportion of PD-L1 treated cells in each cluster and proportion of PD-L1 treated cells in the overall data. FIG. 5F depicts a UMAP showing expression of select genes for monocyte subset and activation markers. FIG. 5G shows a scatter plot showing the effects of exposing in vitro myeloid cells to anti-PD-1 antibody and/or sPD-L1 on Ly6C expression. FIG. 5H shows oxygen consumption rate of myeloid cells treated with PD-L1+/−anti-PD-1 blocking antibody (FIG. 5I) Bar graphs depict calculated values for respiratory parameters.

FIG. 6 shows human PD-1+ blood monocytes: Representative flow cytometry plots to illustrate gating strategy for monocytes (CD11b+CD15−CD19−CD3−).

FIG. 7 shows murine PD-1+ infiltrating brain macrophages: Representative flow cytometry plots to illustrate gating strategy for PD-1+ macrophages (PD1+CD11b+CD45hi).

FIG. 8 shows 28 neuroscore test results for wild type mice: Violin plots comparing the raw neuroscores of sham, untreated, and treated mice at weeks 1, 2, 3, and 4. **** indicated p<0.0004.

FIGS. 9A-B show behavioral and immunologic effects of PD-L1 treatment in sham operated mice. FIG. 9A shows that PD-L1 treated (n=15) and untreated (n=14) mice showed no difference in 28 point scores 24 hours or 7 days after surgery (p=0.837 reflects the association between PD-L1 and have a 28-point neuroscore in the 4^{th} quartile). FIG. 9B shows that PD-L1 treatment did not change the density or PD-1 expression of monocytes in the brain or blood of sham animals. Statistical analyses were performed by Students T-test and Chi-square and Fisher exact tests.

FIGS. 10A-B show PD-L1 expression in treated and untreated mice after MCAO. FIG. 10A shows UMAP of PD-L1 expression. FIG. 10B depicts a volcano plot showing upregulation of PD-L1 (p-value=3.06369328080052e-290 with a log 2 fold change of 2.14749020509468) and PD-L2 (8.8806260334966e-155 with a log 2 fold change of 2.92326622611452) increased in PD-L1 treated cells in cluster 5.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise.

The use of the word “a” or “an” when used in conjunction with the term “comprising” in the claims and/or the specification may mean “one,” but it is also consistent with the meaning of “one or more,” “at least one,” and “one or more than one.”

“Optional” or “optionally” means that the subsequently described event, circumstance, or material may or may not occur or be present, and that the description includes instances where the event, circumstance, or material occurs or is present and instances where it does not occur or is not present.

The word “or” as used herein means any one member of a particular list and also includes any combination of members of that list. The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. “Inhibit,” “inhibiting” and “inhibition” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Treatment” and “treating” refer to administration or application of a therapeutic agent (e.g., PD-1 agonist) to a subject or performance of a procedure or modality on a subject for the purpose of obtaining a therapeutic benefit of a disease or health-related condition. For example, a treatment may include administration of a pharmaceutically effective amount of a PD-1 agonist.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition (e.g., stroke). Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be cerebral edema after a stroke or neuroinflammation after a stroke.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In another aspect, a subject is a human. In some aspects, a subject is a non-human primate. The term does not denote a particular age or sex. Thus, adult, child, adolescent and newborn subjects, as well as fetuses, whether male or female, are intended to be covered.

As used herein, the term “patient” refers to a subject afflicted with a condition, disease or disorder (e.g., cerebral edema after a stroke or neuroinflammation after a stroke). The term “patient” includes human and veterinary subjects. In some aspects of the disclosed methods, the “patient” has been diagnosed with cerebral edema after a stroke or neuroinflammation after a stroke. In some aspects of the disclosed methods, the “patient” has been diagnosed with a need for treatment (e.g. treatment for cerebral edema after a stroke or neuroinflammation after a stroke, of improving gait after stroke, improving sensorimotor deficits after stroke, reducing the number of PD-1 positive monocytes in the brain after a stroke, decreasing intracranial pressure after a stroke, shifting the phenotype of monocytes in the brain from a classical inflammatory subtype to a non-classical subtype after a stroke, limiting or reducing secondary inflammatory injury after a stroke, or reducing a risk of a second or more stroke events in a subject after a stroke), such as, for example, prior to the administering step.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Stroke is the second leading cause of death worldwide (1) and a leading cause of long-term disability V. L. Feigin, et al., Circ Res. 120, 439-448 (2017)). An estimated 87% of strokes are ischemic (S. S. Virani, et al. Circulation. (2020)), and large vessel occlusions (LVOs)—defined as occlusion of the internal carotid artery (ICA), proximal middle cerebral artery (M1, M2), proximal anterior cerebral artery (A1, A2), vertebral artery, basilar artery, or proximal posterior cerebral artery (P1, P2)—account for 20-40% of acute ischemic strokes (K. Malhotra, et al, Front Neurol. 8, 651 (2017); and W. S. Smith, et al., Stroke. 40, 3834-3840 (2009)). Primary injury in acute ischemic stroke results from rapid cell death in the infarct core due to sudden disruption of cerebral perfusion. Secondary injury is initiated when dying neurons in the ischemic core undergo metabolic failure, resulting in cytotoxic edema (D. Liang, et al. Neurosurg Focus. 22, E2 (2007); J. A. Stokum, et al. J Cereb Blood Flow Metab. 36, 513-538 (2016); and J. M. Simard, et al., Lancet Neurol. 6, 258-268 (2007)) and release damage-associated molecular patterns (DAMPs). DAMPs trigger activation, recruitment, and trafficking of immune cells into the ischemic core and penumbra, with maximum accumulation of neutrophils and monocytes during the first three to seven days post-ictus (M. Gelderblom, et al., Stroke. 40, 1849-1857 (2009); and Y. Qiu, et al., Front Immunol. 12, 678744 (2021)). This immune cell infiltration contributes to endothelial dysfunction of the cerebral microvasculature, causes blood brain barrier (BBB) permeabilization, and generates the driving force for vasogenic (trans-vascular) edema. Unlike cytotoxic edema, which is due to early intracellular fluid shifts, vasogenic edema is caused by extracellular water extravasation from the vascular compartment down ion gradients, and results in increased volume or swelling of the brain tissues (J. A. Stokum, et al. J Cereb Blood Flow Metab. 36, 513-538 (2016); and J. M. Simard, et al., Lancet Neurol. 6, 258-268 (2007)). The resulting mass effect within the fixed cranial vault causes elevated intracranial pressure and brain shift, with a high incidence of permanent injury or death (J. Hofmeijer, et al. Cerebrovasc Dis. 25, 176-184 (2008)).

LVOs are associated with larger infarct volumes and contribute to a disproportionately higher rate of post-stroke dependence (61.6%) and mortality (95.6%) (K. Malhotra, et al., Front Neurol. 8, 651 (2017)). The primary intervention is expeditious restoration of cerebral blood flow using intravenous thrombolytics or mechanical thrombectomy to minimize ischemic cell death. The care that follows is supportive. For patients with life-threatening cerebral edema, surgical decompression affords a survival benefit, but does not improve functional outcomes, and carries additional morbidity (J. Lin and J. A. Frontera, Stroke. 52, 1500-1510 (2021)). Attempts to inhibit cerebral inflammation after stroke, including use of steroids, have been unsuccessful due to lack of selectivity and off-target effects (N. Qizilbash, Set al., Cochrane Database Syst Rev., CD000064 (2002)). Targeted anti-inflammatory approaches are of clinical interest to prevent secondary inflammatory injury following LVO.

Immune checkpoints and their ligands are expressed on activated immune cells and protect against aberrant inflammation in healthy tissues or restrain overly robust responses that persist after a threat has been eliminated (D. M. Pardoll, Nat Rev Cancer. 12, 252-264 (2012)). PD-1 is upregulated on immune cells upon activation while its ligands are highly expressed in damaged tissues (D. L. Barber, et al., Nature. 439, 682-687 (2006); and M. E. Keir, et al. Annu. Rev. Immunol. 26, 677-704 (2008)). PD-1 and PD-L1 blocking antibodies have been successfully used in advanced cancers to amplify antitumor immune responses (M. Yi, et al., Mol Cancer. 21, 28 (2022)). More recently, PD-1 agonism to treat chronic inflammation has gained traction as a phase 2a trial of a PD-1 agonist antibody for rheumatoid arthritis generated positive results (E. M. Gravallese and R. Thomas, New England Journal of Medicine. 388, 1905-1907 (2023)). It is not yet clear if PD-1 agonism can be used to treat acute inflammation, which is primarily driven by innate immune cells, however, a growing body of evidence suggests that the PD-1 pathway plays a role in ischemic CNS injury (E. E. Wicks, et al., Front Immunol. 13, 897022 (2022)); and R. Jin, et al., J Leukoc Biol. 87, 779-789 (2010)); however, there are conflicting data regarding the outcomes of PD-1 activation. In a middle cerebral artery occlusion (MCAO) model, PD-1 knockout mice had larger infarcts and worse functional outcomes when compared with wild-type (X. Ren, et al., Stroke. 42, 2578-2583 (2011)). Conversely, PD-L1 blockade decreased infarct volumes and improved outcomes after MCAO (S. Bodhankar, et al., Stroke. 46, 2926-2934 (2015)). These conflicting results may, in part, be due to location of PD-1/PD-L1 interactions as systemic administration of sPD-L1 decreases inflammation after ICH (R. Han, et al., Stroke. 48, 2255-2262 (2017)). It has been shown that PD-1 expression on circulating monocytes in patients with ruptured cerebral aneurysms correlated with cerebral vasospasm, while systemic sPD-L1 administration prevented vasospasm after subarachnoid hemorrhage in an ICA perforation model by inhibiting ingress of PD-1+, Ly6Chi, CCR2hi inflammatory monocytes into the brain (C. M. Jackson, et al., Neurosurgery. 88, 855-863 (2021)). Disclosed herein are methods of using sPD-L1 to activate PD-1 on peripheral monocytes and limiting secondary inflammatory injury after LVO.

### Methods of Treatment

Disclosed herein are methods of reducing or preventing cerebral edema in a subject after a stroke. In some aspects, the methods can comprise: administering systemically a therapeutically effective amount of a PD-1 agonist to the subject. In some aspects, the cerebral edema after stroke can be malignant cerebral edema. In some aspects, the cerebral edema after stroke can be refractory cerebral edema. In some aspects, the subject had an acute ischemic stroke. In some aspects, the subject had a subarachnoid hemorrhage. In some aspects, the acute ischemic stroke was caused by a large vessel occlusion. In some aspects, the large vessel occlusion can be a blockage of the internal carotid artery, vertebral artery, basilar artery, M1, A1, or P1. In some aspects, PD-1 expression on monocytes can be upregulated in the acute period after a stroke. In some aspects, the acute period can be the first 72 hours after symptom onset after a subject has a stroke. In some aspects, the administration of the soluable PD-L1 agonist can activate PD-1 on peripheral monocytes. In some aspects, the activated peripheral monocytes can infiltrate the brain, thereby decreasing the number of PD-1 positive monocytes in the brain.

Disclosed herein are methods of treating neuroinflammation in a subject after a stroke. In some aspects, the methods can comprise: administering systemically a therapeutically effective amount of a PD-1 agonist to the subject. In some aspect, the methods of treating inflammation can result in a decrease in neuroinflammation that can be measured using MRI or assessing various clinical outcomes. In some aspects, the methods can show improvements that can be measured on MRI. For example, T2 and fluid attenuated inversion recovery (FLAIR) can be used. In some aspects, the methods can show improvements in various clinical outcomes. Examples of clinical outcomes include but are not limited to survival, gait, motor function, cognitive function, and functional outcome scores such as Glasgow Outcome Scale (GOS), Montreal Cognitive Assessment, and similar clinical measures of function. In some aspects, administering systemically a therapeutically effective amount of a PD-1 agonist to the subject is expected to decrease expression of Ly6c on blood monocytes, and skew towards a non-inflammatory (M2) phenotype. In some aspects, administering systemically a therapeutically effective amount of a PD-1 agonist to the subject is expected to demonstrate less edema per stroke volume as measured by the ratio of ADC and diffusion weighted imaging to FLAIR/T2 sequences using MRI. In some aspects, the subject had an acute ischemic stroke. In some aspects, the subject had a subarachnoid hemorrhage. In some aspects, the acute ischemic stroke was caused by a large vessel occlusion. In some aspects, the large vessel occlusion can be a blockage of the internal carotid artery, vertebral artery, basilar artery, M1, A1, or P1. In some aspects, PD-1 expression on monocytes can be upregulated in the acute period after a stroke. In some aspects, the acute period can be the first 72 hours after symptom onset after a subject has a stroke. In some aspects, the administration of the PD-1 agonist can activate PD-1 on peripheral monocytes. In some aspects, the activated peripheral monocytes can infiltrate the brain, thereby decreasing the number of PD-1 positive monocytes in the brain.

Disclosed herein are methods of improving gait in a subject after a stroke. In some aspects, the methods can comprise: administering systemically a therapeutically effective amount of a PD-1 agonist to the subject. In some aspects, improvements in gait can be assessed by a physical therapist. For example, an improvement in the subject's ability to stand or walk can be observed. In some aspects, the ability for a subject to step over objects, lift legs, stand on one leg, increase walking speed can be improved after administering systemically a therapeutically effective amount of a PD-1 agonist. In some aspects, the subject had an acute ischemic stroke. In some aspects, the subject had a subarachnoid hemorrhage. In some aspects, the acute ischemic stroke was caused by a large vessel occlusion. In some aspects, the large vessel occlusion can be a blockage of the internal carotid artery, vertebral artery, basilar artery, M1, A1, or P1. In some aspects, PD-1 expression on monocytes can be upregulated in the acute period after a stroke. In some aspects, the acute period can be the first 72 hours after symptom onset after a subject has a stroke. In some aspects, the administration of the PD-1 agonist can activate PD-1 on peripheral monocytes. In some aspects, the activated peripheral monocytes can infiltrate the brain, thereby decreasing the number of PD-1 positive monocytes in the brain.

Disclosed herein are methods of improving sensorimotor deficits in a subject after a stroke. In some aspects, the methods can comprise: administering systemically a therapeutically effective amount of a PD-1 agonist to the subject. Examples of sensorimotor deficits that can be improved include but are not limited to weakness, numbness, proprioception, and gait. In some aspects, the subject had an acute ischemic stroke. In some aspects, the subject had a subarachnoid hemorrhage. In some aspects, the acute ischemic stroke was caused by a large vessel occlusion. In some aspects, the large vessel occlusion can be a blockage of the internal carotid artery, vertebral artery, basilar artery, M1, A1, or P1. In some aspects, PD-1 expression on monocytes can be upregulated in the acute period after a stroke. In some aspects, the acute period can be the first 72 hours after symptom onset after a subject has a stroke. In some aspects, the administration of the PD-1 agonist can activate PD-1 on peripheral monocytes. In some aspects, the activated peripheral monocytes can infiltrate the brain, thereby decreasing the number of PD-1 positive monocytes in the brain.

Disclosed herein are methods of reducing the number of PD-1 positive monocytes in the brain of a subject after a stroke. In some aspects, the methods can comprise: administering systemically a therapeutically effective amount of a PD-1 agonist to the subject. In some aspects, the number of PD-1 positive monocytes in the brain of a subject after a stroke can be reduced by determining an increase or change in the cell phenotype in the blood. For example, the phenotype of monocytes in the brain can change from a classical inflammatory subtype to a non-classical subtype. In some aspects, the classical inflammatory subtype can be a CD14^{hi}, CCR2^{hi}, CD16^{lo} phenotype. In some aspects, the non-classical subtype can be a CD14^{lo}, CX3CR1^{hi}, CD16^{hi}, PD-L1+ phenotype. In some aspects, the subject had an acute ischemic stroke. In some aspects, the subject had a subarachnoid hemorrhage. In some aspects, the acute ischemic stroke was caused by a large vessel occlusion. In some aspects, the large vessel occlusion can be a blockage of the internal carotid artery, vertebral artery, basilar artery, M1, A1, or P1. In some aspects, PD-1 expression on monocytes can be upregulated in the acute period after a stroke. In some aspects, the acute period can be the first 72 hours after symptom onset after a subject has a stroke. In some aspects, the administration of the PD-1 agonist can activate PD-1 on peripheral monocytes. In some aspects, the activated peripheral monocytes can infiltrate the brain, thereby decreasing the number of PD-1 positive monocytes in the brain.

Disclosed herein are methods of decreasing intracranial pressure in a subject after a stroke. In some aspects, the methods can comprise: administering systemically a therapeutically effective amount of a PD-1 agonist to the subject. In some aspects, the decrease in intracranial pressure in a subject after a stroke can be measured using MRI. For example, the decrease in intracranial pressure in a subject after a stroke can be visible on MRI as less mass effect or shift. In some aspects, the methods of decreasing intracranial pressure in a subject after a stroke can be a result of decreasing edema. In some aspects, the decrease in edema can be measured using specific MRI sequences. In some aspects, the MRI sequences can be T-2 weight scans or FLAIR. For example, apparent diffusion coefficient (ADC) and diffusion-weighted imaging (DWI) show stroke volume; and FLAIR and T2 weighted imaging show stroke plus edema. In some aspects, the intracranial pressure can be reduced or prevented by at least 1, 5, 10, 15, 20, 25, 30, 35, 40, 45, 50, 55, 60, 65, 70, 75, 80, 85, 90, 95, 96, 97, 98, 99% or any amount in between compared to untreated stroke or subarachnoid hemorrhage patients with similar degrees of injury. In some aspects, the intracranial pressure can be reduced or prevented by at least 1, 5, 10, 15, 20, 25, 30, 35, 40, 45, 50, 55, 60, 65, 70, 75, 80, 85, 90, 95, 96, 97, 98, 99% or any amount in between compared before or after treatment or to a subject that did not receive the treatment. In some aspects, the subject had an acute ischemic stroke. In some aspects, the subject had a subarachnoid hemorrhage. In some aspects, the acute ischemic stroke was caused by a large vessel occlusion. In some aspects, the large vessel occlusion can be a blockage of the internal carotid artery, vertebral artery, basilar artery, M1, A1, or P1. In some aspects, PD-1 expression on monocytes can be upregulated in the acute period after a stroke. In some aspects, the acute period can be the first 72 hours after symptom onset after a subject has a stroke. In some aspects, the administration of the PD-1 agonist can activate PD-1 on peripheral monocytes. In some aspects, the activated peripheral monocytes can infiltrate the brain, thereby decreasing the number of PD-1 positive monocytes in the brain.

Disclosed herein are methods of shifting the phenotype of monocytes in the brain from a classical inflammatory subtype to a non-classical subtype in a subject after a stroke. In some aspects, the methods can comprise: administering systemically a therapeutically effective amount of a PD-1 agonist to the subject. In some aspects, the classical inflammatory subtype can be a CD14^{hi}, CCR2^{hi}, CD16^{lo} phenotype. In some aspects, the classical inflammatory subtype can be a non-classical subtype is a CD14^{lo}, CX3CR1^{hi}, CD16^{hi}, PD-L1+ phenotype. In some aspects, the subject had an acute ischemic stroke. In some aspects, the subject had a subarachnoid hemorrhage. In some aspects, the acute ischemic stroke was caused by a large vessel occlusion. In some aspects, the large vessel occlusion can be a blockage of the internal carotid artery, vertebral artery, basilar artery, M1, A1, or P1. In some aspects, PD-1 expression on monocytes can be upregulated in the acute period after a stroke. In some aspects, the acute period can be the first 72 hours after symptom onset after a subject has a stroke. In some aspects, the administration of the PD-1 agonist can activate PD-1 on peripheral monocytes. In some aspects, the activated peripheral monocytes can infiltrate the brain, thereby decreasing the number of PD-1 positive monocytes in the brain.

Disclosed herein are methods of limiting or reducing secondary inflammatory injury in a subject after a stroke. In some aspects, the methods can comprise: administering systemically a therapeutically effective amount of a PD-1 agonist to the subject. During an inflammatory cascade after ischemic injury, first, neurons die and release danger signals (DAMPS), which recruit inflammatory immune cells. These inflammatory immune cells release inflammatory cytokines that cause more damage to glia and neurons. As more of these cells die, a disruption in ion gradients occurs that causes water to move into the brain parenchyma (vasogenic edema) which can lead to a secondary inflammatory injury in a subject after a stroke. In some aspects, the subject had an acute ischemic stroke. In some aspects, the subject had a subarachnoid hemorrhage. In some aspects, the acute ischemic stroke was caused by a large vessel occlusion. In some aspects, the large vessel occlusion can be a blockage of the internal carotid artery, vertebral artery, basilar artery, M1, A1, or P1. In some aspects, PD-1 expression on monocytes can be upregulated in the acute period after a stroke. In some aspects, the acute period can be the first 72 hours after symptom onset after a subject has a stroke. In some aspects, the administration of the PD-1 agonist can activate PD-1 on peripheral monocytes. In some aspects, the activated peripheral monocytes can infiltrate the brain, thereby decreasing the number of PD-1 positive monocytes in the brain.

Disclosed herein are methods of reducing a risk of a second or more stroke events in a subject after a stroke. In some aspects, the methods can comprise: administering systemically a therapeutically effective amount of a PD-1 agonist to the subject. In some aspects, the subject had an acute ischemic stroke. In some aspects, the subject had a subarachnoid hemorrhage. In some aspects, the acute ischemic stroke was caused by a large vessel occlusion. In some aspects, the large vessel occlusion can be a blockage of the internal carotid artery, vertebral artery, basilar artery, M1, A1, or P1. In some aspects, PD-1 expression on monocytes can be upregulated in the acute period after a stroke. In some aspects, the acute period can be the first 72 hours after symptom onset after a subject has a stroke. In some aspects, the administration of the PD-1 agonist can activate PD-1 on peripheral monocytes. In some aspects, the activated peripheral monocytes can infiltrate the brain, thereby decreasing the number of PD-1 positive monocytes in the brain.

In some aspects, the subject can be a human. In some aspects, the subject can be any human that has had a stroke. In some aspects, the stroke can be any type or subtype of stroke. Examples of types of stroke included but are not limited to ischemic or hemorrhagic strokes. In some aspects, the subject who has had a stroke can be one who also had delayed time to reperfusion or failed reperfusion. In some aspects, the subject who had had a stroke can be one that had a large territory stroke and/or significant edema. In some aspects, the stroke can be of any size. In some aspects, the stroke can be of any size with associated edema.

In some aspects, PD-1 expression on monocytes can be upregulated in the brain in the acute period after a stroke. In some aspects, the PD-1 expression on monocytes can be upregulated in the brain in the acute period after a stroke occurs within the first 72 hours after symptom onset. In some aspects, the increase in PD-1 expression on monocytes can be upregulated in the periphery in the acute period after a stroke occurs within the first 72 hours after symptom onset.

In some aspects, the signs or symptoms of an acute ischemic stroke or a subarachnoid hemorrhage can be severe headache, loss of consciousness, facial droop, hemiparesis, aphasia, nausea and vomiting, confusion, photophobia, neck stiffness, and the like.

In some aspects, the PD-1 agonist can be a soluble PD-L1 or an analogue thereof. In some aspects, the soluble PD-L1 or the analogue thereof can be a PD-L1 fusion protein. In some aspects, the PD-1 agonist for use according to the embodiments can be any of those described in U.S. Publication No. 2023-0123454, which is incorporated herein by reference for its teaching of fusion proteins including PD-L1 protein and a modified immunoglobulin Fc region.

In some aspects, the PD-L1 fusion protein can include a PD-L1 protein and a modified immunoglobulin Fc region.

In some aspects, the PD-L 1 protein can be an extracellular domain of PD-L1 protein or a fragment thereof. The extracellular domain of the PD-L1 protein can be a polypeptide including an immunoglobulin V like domain (Ig V like domain) of PD-L1 and an immunoglobulin C like domain (Ig C like domain) of PD-L1.

In some aspects, the extracellular domain of the PD-L1 protein can be a protein region exposed outside the cell membrane, and can be a polypeptide consisting of the 196 to 238th amino acids of SEQ ID NO: 1 or a polypeptide consisting of the 19th to 239th amino acids of SEQ ID NO: 1.

In some aspects, the extracellular domain of the PD-L1 protein includes an Ig V like (Ig V, Ig V like) sequence that can be a conserved sequence similar to the amino acid sequence of an immunoglobulin (Ig, immunoglobulin), and the highly conserved Ig V like sequence can be the amino acid sequence of the 68th to 114th amino acids of SEQ ID NO: 1. In addition, it can include an Ig C like (Ig C, Ig C like) sequence, and the highly conserved sequence region can be the amino acid sequence of the 153rd to 210th amino acids of SEQ ID NO: 1. In some aspects, the fragment of the extracellular domain of the PD-L1 protein can include all or a part of the Ig V like domain including the Ig V like sequence of PD-L1.

In some aspects, the Ig V like domain in the extracellular domain of the PD-L1 protein can be a site capable of interacting with PD-1, and can be a polypeptide (SEQ ID NO: 3) consisting of the amino acid sequences of the 19th to 239^{th} amino acids of SEQ ID NO: 1 or a polypeptide consisting of the amino acid sequence of the 21″ to 239th amino acids of SEQ ID NO: 1. In some aspects, it can be a polypeptide (SEQ ID NO: 4) consisting of the amino acid sequence of the 19th to 133rd amino acids of SEQ ID NO: 1 or a polypeptide consisting of the amino acid sequence of the 21st to 133rd amino acids of SEQ ID NO: 1. In some aspects, it can be a polypeptide consisting of the amino acid sequence of the 21st to 114^{th} amino acids of SEQ ID NO: 1 or a polypeptide consisting of the amino acid sequence of the 19th to 114th amino acids of SEQ ID NO: 1. In some aspects, it can be a polypeptide consisting of the amino acid sequence of the 21st to 120th amino acids of SEQ ID NO: 1 or a polypeptide consisting of the amino acid sequence of the 19th to 120th amino acids of SEQ ID NO: 1. In some aspects, it can be a polypeptide (SEQ ID NO: 5) consisting of the amino acid sequence of the 19^{th} to 127th amino acids of SEQ ID NO: 1 or a polypeptide (SEQ ID NO: 6) consisting of the amino acid sequence of the 21st to 127th amino acids of SEQ ID NO: 1. In some aspects, it can be a polypeptide consisting of the amino acid sequence of the 21st to 130th amino acids of SEQ ID NO: 1 or a polypeptide consisting of the amino acid sequence of the 19th to 130th amino acids of SEQ ID NO: 1. In some aspects, it can be a polypeptide consisting of the amino acid sequence of the 21st to 131st amino acids of SEQ ID NO: 1 or a polypeptide consisting of the amino acid sequence of the 19th to 131st amino acids of SEQ ID NO: 1.

In some aspects, the amino acid sequence of human PD-L1 VC-like domain (19-239) can be:

(SEQ ID NO: 3)
FTVTVPKDLYVVEYGSNMTIECKFPVEKQLDLAALIVYWEMEDKNII
QFVHGEEDLKVQHSSYRQRARLLKDQLSLGNAALQITDVKLQDAGVY
RCMISYGGADYKRITVKVNAPYNKINQRILVVDPVTSEHELTCQAEG
YPKAEVIWTSSDHQVLSGKTTTTNSKREEKLFNVTSTLRINTTTNEI
FYCTFRRLDPEENHTAELVIPELPLAHPPNERT.

In some aspects, the amino acid sequence of human PD-L1 V-like domain (19-133) can be:

(SEQ ID NO: 4)
FTVTVPKDLYVVEYGSNMTIECKFPVEKQLDLAALIVYWEMEDKNII
QFVHGEEDLKVQHSSYRQRARLLKDQLSLGNAALQITDVKLQDAGVY
RCMISYGGADYKRITVKVNAP.

In some aspects, the amino acid sequence of human PD-L1 V-like domain (19-127) can be:

(SEQ ID NO: 5)
FTVTVPKDLYVVEYGSNMTIECKFPVEKQLDLAALIVYWEMEDKNII
QFVHGEEDLKVQHSSYRQRARLLKDQLSLGNAALQITDVKLQDAGVY
RCMISYGGADYKRIT.

In some aspects, the amino acid sequence of human PD-L1 V-like domain (21-127) can be:

(SEQ ID NO: 6)
VTVPKDLYVVEYGSNMTIECKFPVEKQLDLAALIVYWEMEDKNIIQF
VHGEEDLKVQHSSYRQRARLLKDQLSLGNAALQITDVKLQDAGVYRC
MISYGGADYKRIT.

In some aspects, when the fragment of the extracellular domain of the PD-L1 protein includes an Ig V like domain or a fragment thereof, it can further include an immunoglobulin C like domain (Ig C like domain) of the extracellular domain of the PD-L1 protein. The Ig C like domain can be a polypeptide consisting of the amino acid sequence of the 133rd to 225th amino acids of SEQ ID NO: 1 or a polypeptide consisting of the amino acid sequence of the 134th to 225th amino acids of SEQ ID NO: 1.

In some aspects, when the fragment of the extracellular domain of the PD-L1 protein includes the Ig V like domain or a fragment thereof, it can further include a polypeptide or a fragment thereof including the Ig C like domain of the extracellular domain of the PD-L1 protein. The polypeptide including the Ig C like domain refers to the extracellular domain of the PD-L 1 protein excluding the Ig V domain, and it can be a polypeptide having the 134th to 239^{th} amino acids of SEQ ID NO: 1 (SEQ ID NO: 7) or a polypeptide having the 134th to 238th amino acids of SEQ ID NO: 1 (SEQ ID NO: 8).

In some aspects, the amino acid sequence of human PD-L1 C-like domain (134-239) can be:

(SEQ ID NO: 7)
YNKINQRILVVDPVTSEHELTCQAEGYPKAEVIWTSSDHQVLSGKTT
TTNSKREEKLFNVTSTLRINTTTNEIFYCTFRRLDPEENHTAELVIP
ELPLAHPPNERT.

In some aspects, the amino acid sequence of human PD-L1 C-like domain (134-238) can be:

(SEQ ID NO: 8)
YNKINQRILVVDPVTSEHELTCQAEGYPKAEVIWTSSDHQVLSGKTT
TTNSKREEKLFNVTSTLRINTTTNEIFYCTFRRLDPEENHTAELVIP
ELPLAHPPNER.

In some aspects, the extracellular domain of the PD-L1 protein or a fragment thereof can be derived from a human or a mouse.

In some aspects, the amino acid sequence of mouse PD-L1 V-like domain (21-127) can be:

(SEQ ID NO: 9)
ITAPKDLYVVEYGSNVTMECRFPVERELDLLALVVYWEKEDEQVIQF
VAGEEDLKPQHSNFRGRASLPKDQLLKGNAALQITDVKLQDAGVYCC
IISYGGADYKRIT.

In some aspects, the extracellular domain of the human PD-L1 protein can be a polypeptide (SEQ ID NO: 3) consisting of the amino acid sequence of the 19th to 239th amino acids of SEQ ID NO: 1, and the extracellular domain of the mouse PD-L1 protein can be a polypeptide consisting of the amino acid sequence of the 19th to 239th amino acids of SEQ ID NO: 2. In some aspects, the extracellular domain of the PD-L1 protein can have about 70%, 75%, 80%, 85%, 90%, 91%, 92%, 93%, 94%, 95%, 96%, 97%, 98%, 99% or more homology to a polypeptide sequence consisting of the amino acid sequence of the 19th to 239th amino acids of SEQ ID NO: 1.

In some aspects, the human PD-L1 protein has 290 amino acid residues and includes the amino acid sequence of SEQ ID NO: 1 (Accession Number: Q9NZQ7). In the amino acid sequence of SEQ ID NO: 1, the 1st to 18^{th} amino acid residues at the N-terminus are signal sequences, and the mature human PD-L1 protein includes the amino acid sequence of the 19th to 290th amino acids of SEQ ID NO: 1. The extracellular domain of the human PD-L1 protein includes the amino acid sequence of the 19th to 238^{th} amino acids of SEQ ID NO: 1 or the 19th to 239th amino acids of SEQ ID NO: 1.

In some aspects, the human PD-L1 protein includes an Ig V like domain which is the 19th to 127th amino acids of SEQ ID NO: 1 and an Ig C like domain which is the 134th to 226^{th} amino acids of SEQ ID NO: 1. The amino acid sequence of the human PD-L1 can be:

(SEQ ID NO: 1)
MRIFAVFIFMTYWHLLNAFTVTVPKDLYVVEYGSNMTIECKFPVEKQ
LDLAALIVYWEMEDKNIIQFVHGEEDLKVQHSSYRQRARLLKDQLSL
GNAALQITDVKLQDAGVYRCMISYGGADYKRITVKVNAPYNKINQRI
LVVDPVTSEHELTCQAEGYPKAEVIWTSSDHQVLSGKTTTTNSKREE
KLFNVTSTLRINTTTNEIFYCTFRRLDPEENHTAELVIPELPLAHPP
NERTHLVILGAILLCLGVALTFIFRLRKGRMMDVKKCGIQDTNSKKQ
SDTHLEET.

The mouse PD-L1 protein is reported to contain 290 amino acids, and it includes the amino acid sequence of SEQ ID NO: 2 (Accession Number: Q9EP73). The 1^{st} to 18th amino acid residues of SEQ ID NO: 2 are signal sequences, and the mature mouse PD-L1 protein includes the amino acid sequence of the 19th to 290 th amino acids of SEQ ID NO: 2. The extracellular domain of the mouse PD-L1 protein includes the amino acid sequence of the 19^{th} to 239th amino acids of SEQ ID NO:2. The mouse PD-L1 protein includes an Ig V like protein having the 19th to 127^{th} amino acids of SEQ ID NO: 2 and an Ig C like domain having the 133rd to 224th amino acids of SEQ ID NO: 2. The amino acid sequence of mouse PD-L1 can be:

(SEQ ID NO: 2)
MRIFAGIIFTACCHLLRAFTITAPKDLYVVEYGSNVTMECRFPVERE
LDLLALVVYWEKEDEQVIQFVAGEEDLKPQHSNFRGRASLPKDQLLK
GNAALQITDVKLQDAGVYCCIISYGGADYKRITLKVNAPYRKINQRI
SVDPATSEHELICQAEGYPEAEVIWTNSDHQPVSGKRSVTTSRTEGM
LLNVTSSLRVNATANDVFYCTFWRSQPGQNHTAELIIPELPATHPPQ
NRTHWVLLGSILLFLIVVSTVLLFLRKQVRMLDVEKCGVEDTSSKNR
NDTQFEET.

In some aspects, the extracellular domain of the PD-L1 protein can include the entirety of an Ig V like domain or a fragment thereof. In addition, the fragment of the extracellular domain of the PD-L1 protein can further include an Ig C like domain or a polypeptide including an Ig C like domain (the extracellular domain of PD-L1 excluding the Ig V like domain).

In some aspects, the extracellular domain of the PD-L1 protein or a fragment thereof can include variously modified proteins or peptides. The modification may be performed by substituting, deleting or adding one or more proteins to the wild-type PD-L1 protein as long as the function of PD-L1 is not altered. These various proteins or peptides can have 70%, 75%, 80%, 85%, 90%, 91%, 92%, 93%, 94%, 95%, 96%, 97%, 98% or 99% homology to the wild-type protein.

As used herein, the term “extracellular domain of PD-L1 protein” can also be used as a concept including “the extracellular domain of PD-L 1 protein and a fragment thereof.”

As used herein, the terms “protein”, “polypeptide”, and “peptide” can be used interchangeably unless otherwise specified.

As used herein, the terms “PD-L1 fusion protein” and “PD-L1-modified immunoglobulin Fc region fusion protein” refer to a fusion protein in which PD-L1 protein, the extracellular domain of PD-L 1 protein or a fragment thereof is linked to a modified immunoglobulin Fc region.

In some aspects, the PD-L1 protein can be fused to the N-terminus or C-terminus of a modified immunoglobulin Fc region, and preferably, the PD-L1 protein can be fused to the N-terminus of a modified immunoglobulin Fc region. In some aspects, the PD-L1 protein can be linked to the immunoglobulin Fc region by a linker peptide. In some aspects, the linker can include GGGSGGS (SEQ ID NO: 10), AAGSGGGGGSGGGGSGGGGS (SEQ ID NO: 17), GGSGG (SEQ ID NO: 18), GGSGGSGGS (SEQ ID NO: 19), GGGSGG (SEQ ID NO: 20), (G4S)n (n is an integer from 1 to 10), (GGS)n (n is an integer from 1 to 10), (GS)n (n is an integer from 1 to 10), (GSSGGS)n (n is an integer from 1 to 10; GSSGGS (SEQ ID NO: 36)), KESGSVSSEQLAQFRSLD (SEQ ID NO: 21), EGKSSGSGSESKST (SEQ ID NO: 22), GSAGSAAGSGEF (SEQ ID NO: 23), (EAAAK)n (n is an integer from 1 to 10; EAAAK (SEQ ID NO: 37)), CRRRRRREAEAC (SEQ ID NO: 24), A(EAAAK)₄ALEA(EAAAK)₄A (SEQ ID NO: 38), GGGGGGGG (SEQ ID NO: 25), GGGGGG (SEQ ID NO: 26), AEAAAKEAAAAKA (SEQ ID NO: 27), PAPAP (SEQ ID NO: 28), (Ala-Pro)n (n is an integer from 1 to 10), VSQTSKLTRAETVFPDV (SEQ ID NO: 29), PLGLWA (SEQ ID NO: 30), TRHRQPRGWE (SEQ ID NO: 31), AGNRVRRSVG (SEQ ID NO: 32), RRRRRRRR (SEQ ID NO: 33), GFLG (SEQ ID NO: 34), GSSGGSGSSGGSGGGDEADGSRGSQKAGVDE (SEQ ID NO: 35) and the like. In some aspects, PD-L1 and the immunoglobulin Fc region can be linked by a linker peptide consisting of the amino acid sequence of GGGSGGS (SEQ ID NO: 10). When the PD-L1 protein and the immunoglobulin Fc region are linked using the linker peptide, the activity, stability and productivity of the fusion protein can be optimized.

In some aspects, the PD-L1 fusion protein can be in dimer form.

In some aspects, the modified immunoglobulin Fc region can consist of the amino acid sequence of SEQ ID NO: 11 (“hyFC”). The modified immunoglobulin Fc domain (SEQ ID NO: 11) is a hybrid type of human IgD Fc and human IgG4 Fc, and is characterized by including an IgG1 hinge region (SEQ ID NO: 16) composed of 8 amino acids. In some aspects, the hinge region can include the IgD hinge region of SEQ ID NO: 14 as a control.

In some aspects, the amino acid sequence of the IgD hinge (from hyFc5) can be: RNTGRGGEEKKKEKEKEEQEERETKTPECP (SEQ ID NO: 14). In some aspects, the amino acid sequence of the IgG1 hinge (from modified Fc region) can be: THTCPPCP (SEQ ID NO: 16). In some aspects, the amino acid sequence of the PD-L1 fusion protein comprising the IgD hinge (PD-L1-IgD/hyFc) can be:

(SEQ ID NO: 15)
FTVTVPKDLYVVEYGSNMTIECKFPVEKQLDLAALIVYWEMEDKNII
QFVHGEEDLKVQHSSYRQRARLLKDQLSLGNAALQITDVKLQDAGVY
RCMISYGGADYKRITVKVNAPYNKINQRILVVDPVTSEHELTCQAEG
YPKAEVIWTSSDHQVLSGKTTTTNSKREEKLFNVTSTLRINTTTNEI
FYCTFRRLDPEENHTAELVIPELPLAHPPNERTRNTGRGGEEKKKEK
EKEEQEERETKTPECPSHTQPLGVFLFPPKPKDTLMISRTPEVTCVV
VDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEE
MTKNQVSLTCLVKGFYPSDIAVEWESNGQPENNYKTTPPVLDSDGSF
FLYSRLTVDKSRWQEGNVFSCSVMHEALHNHYTQKSLSLSLGK.

In some aspects, the amino acid of the immunoglobulin Fc region can be:

(SEQ ID NO: 11)
THTCPPCPSHTQPLGVFLFPPKPKDTLMISRTPEVTCVVVDVSQEDP
EVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSL
TCLVKGFYPSDIAVEWESNGQPENNYKTTPPVLDSDGSFFLYSRLTV
DKSRWQEGNVFSCSVMHEALHNHYTQKSLSLSLGK.

In some aspects, the PD-L1 fusion protein can consist of or comprise the amino acid sequence of SEQ ID NO: 12 or SEQ ID NO: 13.

In some aspects, the amino acid sequence of PL-L1 fusion protein (sPD-L1-hyFc) can be:

(SEQ ID NO: 12)
FTVTVPKDLYVVEYGSNMTIECKFPVEKQLDLAALIVYWEMEDKNII
QFVHGEEDLKVQHSSYRQRARLLKDQLSLGNAALQITDVKLQDAGVY
RCMISYGGADYKRITVKVNAPYNKINQRILVVDPVTSEHELTCQAEG
YPKAEVIWTSSDHQVLSGKTTTTNSKREEKLFNVTSTLRINTTTNEI
FYCTFRRLDPEENHTAELVIPELPLAHPPNERTGGGSGGSTHTCPPC
PSHTQPLGVFLFPPKPKDTLMISRTPEVTCVVVDVSQEDPEVQFNWY
VDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGF
YPSDIAVEWESNGQPENNYKTTPPVLDSDGSFFLYSRLTVDKSRWQE
GNVFSCSVMHEALHNHYTQKSLSLSLGK.

In some aspects, the PD-L1 fusion protein can have about 70%, 75%, 80%, 85%, 90%, 91%, 92%, 93%, 94%, 95%, 96%, 97%, 98%, 99% or more homology to the amino acid sequence of SEQ ID NO: 12.

In some aspects, the amino acid sequence of PL-L1 fusion protein (PD-L1-GS/IgG1/hyFc) can be:

(SEQ ID NO: 13)
FTVTVPKDLYVVEYGSNMTIECKFPVEKQLDLAALIVYWEMEDKNII
QFVHGEEDLKVQHSSYRQRARLLKDQLSLGNAALQITDVKLQDAGVY
RCMISYGGADYKRITVKVNAPGGGSGGSTHTCPPCPSHTQPLGVFLF
PPKPKDTLMISRTPEVTCVVVDVSQEDPEVQFNWYVDGVEVHNAKTK
PREEQFNSTYRVVSVLTVLHQDWLNGKEYKCKVSNKGLPSSIEKTIS
KAKGQPREPQVYTLPPSQEEMTKNQVSLTCLVKGFYPSDIAVEWESN
GQPENNYKTTPPVLDSDGSFFLYSRLTVDKSRWQEGNVFSCSVMHEA
LHNHYTQKSLSLSLGK.

In some aspects, the PD-1 agonist can be an antibody or antigen-binding fragment thereof.

In some aspects, the PD-1 agonist can be PD-L1 protein or a derivative thereof. In some aspects, the PD-1 agonist can be a small molecule. In some aspects, the PD-1 agonist can be an antibody, a small molecule, or a fusion peptide.

The compositions described herein can be formulated to include a therapeutically effective amount of a PD-1 agonist described herein. Therapeutic administration encompasses prophylactic applications (e.g., or preventing cerebral edema in a subject after a stroke or preventing a subsequent stroke). Based on genetic testing and other prognostic methods, a physician in consultation with their patient can choose a prophylactic administration where the patient has a clinically determined predisposition or increased susceptibility (in some cases, a greatly increased susceptibility) to cerebral edema or ischemic stroke.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with cerebral edema or after a stroke (e.g., an acute ischemic stroke or a subarachnoid hemorrhage), neuroinflammation, gait deficits, sensorimotor deficits, increases number of PD-1 positive monocytes in the brain, increases intracranial pressure, at risk for a secondary inflammatory injury, at risk for a second or more stroke events, or a predominant classical inflammatory subtype of monocytes in the brain, or one or more symptoms of acute ischemic stroke or a subarachnoid hemorrhage in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the disease, disorder, condition or injury is delayed, hindered, or prevented, or the disease, disorder, condition or injury or a symptom of the disease, disorder, condition or injury is ameliorated or its frequency can be reduced. One or more of the symptoms can be less severe. Recovery can be accelerated in an individual who has been treated. For example, treatment of acute ischemic stroke or a subarachnoid hemorrhage or cerebral edema may involve, for example, a reduction in neuroinflammation, a decrease intracranial pressure, a reduction in the number of PD-1 positive monocytes in the brain, or a shift the phenotype of monocytes in the brain from a classical inflammatory subtype to a non-classical subtype.

In some aspects, the PD-1 agonist can be administered any time after the subject had a stroke. In some aspects, the PD-1 agonist can be administered Is, 10 s, 20 s, 30 s, 40 s, 50 s, 60 s or any time in between after a subject had a stroke. In some aspects, the PD-1 agonist can be administered 1 minute, 5 minutes, 10 minutes, 15 minutes, 20 minutes, 25 minutes, 30 minutes, 35 minutes, 40 minutes, 45 minutes, 50 minutes, 55 minutes, 60 minutes or any time in between after a subject had a stroke. In some aspects, the PD-1 agonist can be administered 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, 72 hours, or any time in between after a subject had a stroke. In some aspects, the PD-1 agonist can be administered 1 day, 2 days, 3 days, 4 days, 5 days, 6 days, 7 days or any time in between after a subject had a stroke. In some aspects, the PD-1 agonist can be administered 1 week, 2 weeks, 3 weeks, 4 weeks, or any time in between after a subject had a stroke. In some aspects, the PD-1 agonist can be administered within the first 72 hours after a subject had a stroke. In some aspects, the PD-1 agonist can be administered within 0-2 weeks after a subject had a stroke. In some aspects, the PD-1 agonist can be administered between 1 s and 72 hours after the subject had the stroke.

In some aspects, the PD-1 agonist can be administered with at least a second therapeutic agent. The methods and compositions, including combination therapies, can enhance the therapeutic or protective effect, and/or increase the therapeutic effect to any of the PD-1 agonists described herein.

The PD-1 agonists can be administered before, during, after, or in various combinations relative to a second therapeutic agent or therapy. The administrations may be in intervals ranging from concurrently to minutes to days to weeks. In aspects where the PD-1 agonists are provided to a patient separately from a second therapeutic agent or therapy, one would generally ensure that a significant period of time did not expire between the time of each delivery, such that the two compounds would still be able to exert an advantageously combined effect on the patient. In such instances, it is contemplated that one may provide a patient with the PD-1 agonist and the second therapeutic agent or therapy within about 12 to 24 or 72 h of each other and, more particularly, within about 6-12 h of each other. In some situations, it may be desirable to extend the time period for treatment significantly where several days (2, 3, 4, 5, 6, or 7) to several weeks (1, 2, 3, 4, 5, 6, 7, or 8) lapse between respective administrations.

In some aspects, a course of treatment can last between 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there can be a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below a syntenin-1 inhibitor or a syndecan-1 inhibitor is “A” and a second therapeutic agent is “B”:

A/B/A B/A/B B/B/A A/A/B A/B/B B/A/A A/B/B/B B/A/B/B B/B/B/A B/B/A/B
A/A/B/B A/B/A/B A/B/B/A B/B/A/A B/A/B/A B/A/A/B A/A/A/B B/A/A/A A/B/A/A
A/A/B/A.

Administration of any compound or therapy disclosed herein to a patient will follow general protocols for the administration of such compounds, taking into account the toxicity, if any, of the agents. Therefore, in some aspects there can be a step of monitoring toxicity that can be attributable to combination therapy.

In some aspects, the second therapeutic agent can be a nimodipine, hypertonic saline, mannitol, or a combination thereof. For example, when treating a subject with a subarachnoid hemorrhage, the second therapeutic agent can be a nimodipine. In some aspects, when treating a subject with a large vessel occlusion, the second therapeutic agent can be hyperonic saline and/or mannitol.

The compositions described herein used in the disclosed methods can be formulated to include a therapeutically effective amount of the PD-1 agonist disclosed herein. In some aspects, the PD-1 agonist thereof disclosed herein can be contained within a pharmaceutical formulation. In some aspects, the pharmaceutical formulation can be a unit dosage formulation.

The therapeutically effective amount or dosage of any of the PD-1 agonists used in the methods as disclosed herein applied to mammals (e.g., humans) can be determined by one of ordinary skill in the art with consideration of individual differences in age, weight, sex, the severity of the subject's symptoms, and the particular composition or route of administration selected, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the symptoms), the age and physical characteristics of the subject and other considerations known to those of ordinary skill in the art. Dosages can be established using clinical approaches known to one of ordinary skill in the art. A therapeutically effective dosage of the PD-1 agonist can result in a decrease in severity of one or more disease symptoms, an increase in frequency and duration of disease symptom-free periods, or a prevention of impairment or disability due to the disease affliction. As disclosed therein, in some aspects a therapeutically effective amount of a PD-1 agonist can reduce or prevent cerebral edema, treat or reduce neuroinflammation, improve gait, improve sensorimotor deficits, reduce the number of PD-1 positive monocytes in the brain, decrease intracranial pressure, shift the phenotype of monocytes in the brain from a classical inflammatory subtype to a non-classical subtype, limit or reduce secondary inflammatory injury, reduce a risk of a second or more stroke events or otherwise reduce or ameliorate one or more symptoms in a subject after a stroke.

The duration of treatment with any composition in the methods disclosed herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The total effective amount of the PD-1 agonist as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

### Pharmaceutical Compositions

As disclosed herein, are pharmaceutical compositions, comprising a PD-1 agonist disclosed herein. As disclosed herein, are pharmaceutical compositions, comprising a PD-1 agonist and a pharmaceutical acceptable carrier described herein. In some aspects, the PD-1 agonist can be formulated for systemic administration. In some aspects, the PD-1 agonist can be formulated for intravenous, intraperitoneal, or oral administration. In some aspects, the PD-1 agonist can be formulated for oral or parental administration. In some aspects, the parental administration can be intravenous, subcutaneous, intramuscular or direct injection. In some aspects, the PD-1 agonist can be administered intramuscularly, intravenously, subcutaneously, orally, topically, transdermally, or sublingually. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E. W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes any of the PD-1 agonists dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

### Articles of Manufacture

The composition described herein can be packaged in a suitable container labeled, for example, for use as a therapy to treating or preventing rheumatoid arthritis or any of the methods disclosed herein. Accordingly, packaged products (e.g., sterile containers containing the composition described herein and packaged for storage, shipment, or sale at concentrated or ready-to-use concentrations) and kits, including at least one or more of the PD-1 agonists as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compositions can be ready for administration (e.g., present in dose-appropriate units), and may include a pharmaceutically acceptable adjuvant, carrier or other diluent. Alternatively, the compositions can be provided in a concentrated form with a diluent and instructions for dilution.

### EXAMPLES

It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1. Soluble PD-L1 Reprograms Blood Monocytes to Prevent Cerebral Edema and Facilitate Recovery after Ischemic Stroke

Acute cerebral ischemia triggers a profound inflammatory response. While macrophages polarized to an M2-like phenotype clear debris and facilitate tissue repair, aberrant or prolonged macrophage activation is counterproductive to recovery. The inhibitory immune checkpoint Programmed Cell Death Protein 1 (PD-1) is upregulated on macrophage precursors (monocytes) in the blood after acute cerebrovascular injury. To investigate the therapeutic effects of PD-1 activation, circulating monocytes from patients were immunophenotypes and the results show that PD-1 expression was upregulated in the acute period after stroke. Murine studies using a temporary middle cerebral artery (MCA) occlusion (MCAO) model showed that intraperitoneal administration of soluble Programmed Death Ligand-1 (sPD-L1) significantly decreased brain edema and improved overall survival. Mice receiving sPD-L1 also had higher performance scores short-term, and more closely resembled sham animals on assessments of long-term functional recovery. These clinical and radiographic benefits were abrogated in global and myeloid-specific PD-1 knockout animals, confirming PD-1+ monocytes as the therapeutic target of sPD-L1. Single-cell RNA sequencing revealed that treatment skewed monocyte maturation to a non-classical Ly6Clo, CD43hi, PD-L1+ phenotype. These data support peripheral activation of PD-1 on inflammatory monocytes as a therapeutic strategy to treat neuroinflammation after acute ischemic stroke.

Materials and Methods. Study Design. This study was designed to investigate the anti-inflammatory effects of sPD-L1 in the setting of acute ischemic stroke. Using a well-established murine model of LVO and MCA distribution infarct, the role of PD-1 expressing monocytes in neuroinflammation and cerebral edema and as targets of therapeutic intervention was evaluated. High-resolution MRI, wet versus dry weight measurements, flow cytometry, standardized behavioral tests, scRNA-seq, and a Seahorse bioanalyzer were used. Human blood samples were collected.

Human patient sample collection. A total of 14 adult (>18 years) patients who presented to the Johns Hopkins Hospital between January 2021 and January 2023 with acute LVO of the middle cerebral artery were enrolled into the study. Recorded characteristics included patient sex, time of last known well, presentation time, the Alberta stroke program early CT score (ASPECTS) on arrival, LVO location including vessel involved and laterality, National Institutes of Health Stroke Scale (NIHSS) score on arrival, whether the patient underwent mechanical thrombectomy, timing of recanalization, Thrombolysis in Cerebral Infarction (TICI) score, and total infarct volume and edema volume. Volumetric measurements were calculated using manual segmentation. Patients with pre-existing active systemic infections such as COVID-19 or hepatitis, or who underwent emergent decompressive craniectomy or any surgical intervention other than mechanical thrombectomy during the period of blood sample collection were excluded. Patients who presented with small-vessel strokes, acute strokes in territories other than the MCA, or who did not undergo a follow-up MRI with T2 and DWI/ADC sequences were excluded from the study. Brain edema and infarct volumes were calculated from manually traced ROIs of the T2 and DWI/ADC images, respectively. Brain regions of interest (ROI) were analyzed using CARESTREAM Vue PACS software (Carestream Health Inc, Rochester, NY). ROIs were manually segmented, and areas within each slice were automatically calculated, summed across the slices, and multiplied by slice thickness to calculate total volume.

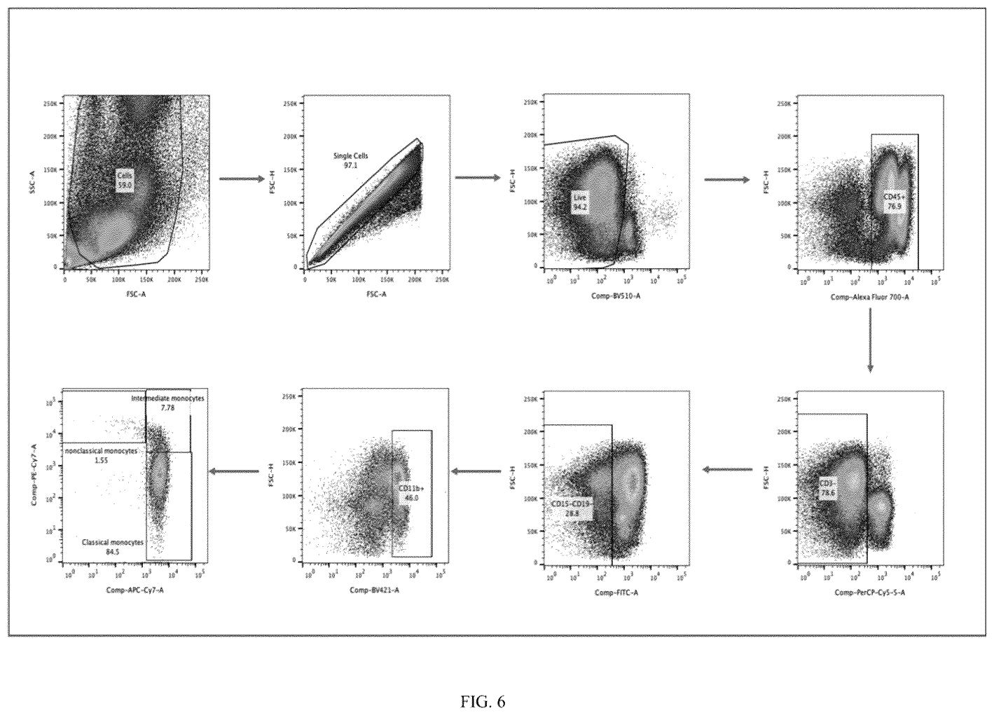

Flow cytometric analysis of human monocytes. Peripheral blood samples were collected at 6, 12, and 24 hours after initial presentation, then daily for up to seven days by venipuncture or indwelling venous or arterial line. Leukocytes were isolated from whole blood samples by Ficoll (Sigma-Aldrich) density gradient centrifugation. Immune cell isolates were washed and resuspended in phosphate buffered saline (PBS) and stained for CD3, CD45, CD11b, CD19, CD15, CD14, CD16, CD11c, CD8, and PD-1. Data were acquired using a FACSAria (BD) and analyzed using FlowJo (Treestar) (Table 1). The gating strategy is shown in FIG. 6.

TABLE 1
28-point score quartile breakdown in MCAO versus
MCAO + PD-L1 wild-type mice, with P-value
comparisons for falling in the highest quartile.

| | 1 | 2 | 3 | 4 | P-value |
|---|---|---|---|---|---|
| | Week 2 Quartiles | | | | |
| MCAO | 7 (21) | 6 (18) | 7 (21) | 13 (39) | 0.284 |
| MCAO + PD-L1 | 7 (23) | 7 (23) | 9 (29) | 8 (26) | |
| | Week 3 | | | | |
| MCAO | 8 (24) | 4 (12) | 11 (33) | 10 (30) | 0.524 |
| MCAO + PD-L1 | 7 (23) | 4 (13) | 13 (42) | 7 (23) | |
| | Week 4 | | | | |
| MCAO | 6 (19) | 3 (9) | 17 (53) | 6 (19) | 0.187 |
| MCAO + PD-L1 | 6 (19) | 7 (23) | 8 (26) | 10 (32) | |

Mice. Male C57BL/6J (8-10 weeks) wild-type mice (Jackson M E) were maintained and housed in a specific-pathogen-free (SPF) vivarium, which was temperature- and humidity-controlled (21±3° C., 50±10%), under a 12 h light/dark cycle. The animals had access to the same food and water ad libitum throughout the entire study. Male 8-10 week-old mice were used for the described studies. Animals were euthanized according to humane endpoints, including central nervous system disturbances, hunched posture, lethargy, weight loss, and inability to ambulate. C57BL/6J wild-type (WT) mice were purchased from The Jackson Laboratory (Bar Harbor, Maine). PD-1^{-/-} (B6·Cg-Pdcd1tm1.1Shr/J) mice were purchased from The Jackson Laboratory (Bar Harbor, Maine). The mice were bred at the Johns Hopkins University Animal Facility. PD-1f/fLysMcre mice were generated by mating Pdcd1flox/flox (PD-1f/f) mice on a C57BL/6 background with LysMcre mice (B6.129P2-Lyz2tm1 (cre)Ifo/J) or CD4cre mice (B6·Cg-Tg(Cd4-cre)1Cwi/BfluJ) (L. Strauss, et al. Sci Immunol. 5, eaay 1863 (2020).). Genetic ablation was confirmed by genotyping of pups through Transnetyx (Memphis, TN).

Transient MCAO. Mice were induced and maintained with 3% and 1.5% isoflurane, respectively, in enriched O2 using a vaporizer. Rectal temperature was maintained at ≈37° C. The mouse was placed prone and a right-sided scalp incision was made midway between the lateral canthus and the anterior pinna. A laser Doppler (Perimed PF 6000) flow probe was attached perpendicularly to the right lateral parietal skull. The mouse was then positioned supine. A midline cervical incision was made under an operating microscope, and under high magnification, the right external carotid artery (ECA), right common carotid artery (CCA) and right internal carotid artery (ICA) were dissected free of the surrounding tissue. The ECA was permanently ligated with a 7-0 silk suture, and the ICA and CCA were occluded with 3 mm temporary aneurysm clips (Aesculap). A silicone-coated 6-0 nylon suture (Doccol, 602223PK10Re) was passed into the lumen of the ECA and guided into the ICA. The temporary clip on the ICA was removed and the suture was advanced past the skull base until resistance was met. MCA territory flow occlusion was verified by a sustained 80% drop of baseline cortical perfusion values. After 45 minutes of occlusion, the suture was withdrawn to allow reperfusion of the vessel and the incisions were closed. Any animal that experienced technical complications during the induction of MCAO, such as excessive bleeding, prolonged operation time, or a less than 80% drop of baseline cortical perfusion values as measured by laser Doppler were excluded. Survival rates were documented daily for up to 30 days after surgery. Sham-operated mice underwent the same surgical treatment, with the arteries exposed and the ECA ligated, but without filament insertion into the MCA.

PD-L1 administration. Recombinant Mouse B7-H1 (PD-L1, CD274)-Fc Chimera (carrier-free) (Biolegend, Cat 758208) was diluted in sterile PBS such that 50 μg of PD-L1 in a total volume of 200 mL was administered by intraperitoneal injection 1 hour and 24 hours after reperfusion.

Brain water content. Mice were deeply anesthetized and killed by decapitation at 48 hours after MCAO, after which the brain was quickly removed and heated for 72 hours at 100° C. in a drying oven (Vacutherm, Fisher Scientific). Brain water content was measured by comparing wet-to-dry ratios (R. F. Keep, et al., Transl Stroke Res. 3, 263-265 (2012)). Tissues were weighed with a scale to within 0.001 mg. Tissue water content was then calculated as (1) Water content=(wet weight−dry weight)/dry weight, and (2) % Water content=100×(wet weight−dry weight)/wet weight.

MRI acquisition and analysis. High-resolution MRI was performed 72 hours after MCAO surgery on an 11.7T Ultra Shielded Bruker Biospec system (Bruker, Ettlingen, Germany) with a horizontal bore. Images were acquired using a 72-mm quadrature volume resonator as a transmitter, and a four-element (2×2) phased-array mouse-head coil as a receiver. The animal was fixed on a plastic holder and anesthetized with 3-4% isoflurane for induction, and 1-2% for maintenance. The respiratory rate was monitored using a pressure pad and a thermostatically controlled heating reel was used to keep the body temperature at 37 degrees. Apparent diffusion coefficient (ADC), spin echo planar imaging diffusion-weight imaging (SE-EPI-DWI), and T2 MRI sequences were acquired.

Brain regions of interest (ROI) were analyzed using Paravision 6.01 software (Bruker, Ettlingen, Germany). Brain edema and infarct volumes were calculated from manually traced ROIs of the T2 and DWI/ADC images, respectively. Areas within each slice were calculated, summed across the slices, and multiplied by slice thickness to calculate total volume.

MRI acquisition parameters. The parameters for T2w images were 36 interleaved coronal 0.5 mm slices that were obtained with a FOV (field-of-view) of 1.5×1.5 cm, image size 128×128, TR of 4,000 ms, TE of 50 ms, 4 averages, and band width 50 kHz. Fat saturation and triggering were used to suppress signal from fat and minimize breathing motion artifacts, respectively. Bruker standard RARE sequence was used with a factor=8.

The parameters for EPI-DWI Images of 36 interleaved coronal 0.5 mm slices were obtained with a FOV (field-of-view) of 1.5×1.5 cm, image size 128×128, TR of 5,000 ms, TE of 27.76 ms, 4 segments, and a band width 40 kHz, and b-value of 1,500 s/mm2 with 16 gradient directions. EPI-DTI. Fat saturation and triggering were used to suppress signal from fat and minimize breathing motion artifacts, respectively.

Immune cell isolation. To isolate brain-infiltrating immune cells, brains were harvested 48 hours post-MCAO. The brains were mechanically homogenized and filtered on ice, then resuspended in 5 mL 70% Percoll, layered below 7 mL of 37% Percoll and centrifuged at 2000 rpm for 20 mins at room temperature. The cell layer at the 37%/70% interface was collected and washed with PBS.

Flow cytometric analysis of murine monocytes. To analyze surface markers, immune cells were pre-treated with Fc Block (anti-CD16/32) (BD Biosciences), washed, and stained with Live-Dead Aqua (Invitrogen) at 4° C. for 30 minutes. Cells were then washed and stained at 4° C. for 30 minutes with CD45, CD11b, CD3, CD8, Ly6C, Ly6G, CCR2, and PD-1 (Table 2 for antibody clones and dilutions). Data were acquired using a FACSAria (BD) flow cytometer and analyzed using FlowJo software (TreeStar). Nonviable cells were excluded by forward versus side scatter analysis and Live-Dead Aqua (Invitrogen) staining. The gating strategy is shown in FIG. 7.

TABLE 2
Antibodies used for flow cytometry of murine tissue samples.

| Antibody (Flow) | Clone | Fluorophore | Dilution | Company |
|---|---|---|---|---|
| CD3 | 145-2C11 | PercpCy5.5 | 5:200 | Biolegend |
| CD45 | 30-F11 | APC-Cy7 | 1:200 | Biolegend |
| CD11b | M1/70 | AF700 | 1:200 | Biolegend |
| CD11c | N418 | APC | 1:200 | Biolegend |
| Ly6C | HK1.4 | FITC | 1:200 | Biolegend |
| Ly6G | 1A8 | BV650 | 1:200 | Biolegend |
| CD43 | S11 | PE-Cy7 | 1:200 | Biolegend |
| PD-1 | RMP1-30 | BV421 | 1:200 | Biolegend |
| CCR2 | SA203G11 | PE | 1:200 | Biolegend |

Behavioral tests. Functional outcomes were assessed using a 28-point neuroscore at 1, 2, 3, and 4 weeks after surgery by a blinded, experienced experimenter. Mouse gait was assessed using a motorized treadmill (DigiGait Imaging System, Mouse Specifics, Boston, MA) and analyzed using DigiGait analysis software (Mouse Specifics, Inc.). Exploratory and locomotor activity of mice was measured by open field test at 4 weeks post-surgery. Behavioral tests were performed in a well-lit environment. Animals were acclimated in the testing room for an hour before testing, and the testing areas were thoroughly cleaned between animals.

28-point neuroscore. Functional deficits were assessed with the 28-point neuroscore test (A. Encarnacion, et al., J Neurosci Methods. 196, 247-257 (2011)). The cumulative maximum score of 28 point is based on eleven individual tests: (1) circling behavior (maximum 4 points), (2) motility (maximum 3 points), (3) general condition (maximum 3 points), (4) paw placement (maximum 4 points), (5) righting reflex (maximum 1 point), (6) pulling-up on a horizontal bar (maximum 3 points), (7) climbing on an inclined plane (maximum 3 points), (8) grip strength (maximum 2 points), (9) contralateral reflex (maximum 1 point), (10) forepaw reaching (maximum 2 points), and (11) contralateral rotation when held by the tail (maximum 2 points). Scoring was determined on a scale from 0 (worst functional and behavioral outcome) to the maximum score of 28 points for healthy animals (A. Encarnacion, et al., J Neurosci Methods. 196, 247-257 (2011); and K. Arkelius, et al., Sci Rep. 10, 1-12 (2020)).

Open field. The open field test was used to assess locomotor activity and exploration habits of mice in a relatively large, circular enclosure. A circular open field arena (40 cm diameter) was created using a white PVC cylinder (0.41 m diameter, 0.3 m height) and placed in a quiet, well-lit room (130 lm). The open field was divided into three concentric circles—center, neutral, and peripheral zones. Each mouse was placed against the wall of the arena and allowed to explore freely for 15 minutes. Anymaze™ video-tracking software was used to record and measure behavior (L. L. Jantzie, et al., Front Neurol. 9, 233 (2018)).

Gait assessment. Gait analysis was performed using the DigiGait Imaging System (Mouse Specifics Inc., Boston, MA) (J. Vincelette, et al., Arthritis Res Ther. 9, R123 (2007)). Digital video images of the animal's underside were collected with a high-speed video camera placed below the transparent belt of a motorized treadmill (L. L. Jantzie, et al., J Neuroinflammation. 11, 131 (2014); and H. Koshimizu, et al., PLOS One. 9, e89584 (2014)). The settings, such as the camera focus, lighting, and belt speed, were optimized before testing. Mice were forced to walk or run at a fixed velocity (L. Yang, et al., Front Neurol. 13, 834329 (2022)), which was preset to 10 cm/s at week 1 and 15 cm/s at week 3. The mice were acclimated to the enclosure and the walking speed before recording for analysis. DigiGait analysis software v.12.2 (Mouse Specifics, Inc.) identified each paw as the mouse walked on the belt. Video recordings in which a mouse walked without stumbling and did not contact the walls or bumpers were used for measurements and analyses. Poor quality video or gait analysis tracings were excluded. The areas of each paw relative to the belt and camera were calculated throughout each stride. When plotted as a function of time, the areas provided a dynamic gait signal that denoted the braking, swing, and propulsion components of each stride. Stride, stance, and swing durations were also calculated from these signals. Additional variables were calculated by the software to describe other aspects of gait, including paw angle and relative positions of the paws to each other and to the midline (J. Vincelette, et al., Arthritis Res Ther. 9, R123 (2007); and E. R. Berryman, et al., J Musculoskelet Neuronal Interact. 9, 89-98 (2009)).

Single Cell RNA Sequencing and Data Analysis. Blood was collected by cardiac puncture from anesthetized mice 48 hours after MCAO. The blood was centrifuged at 300 G for 5 minutes and resuspended in ACK lysing buffer (Life) three times for five minutes on ice to lyse nucleated red blood cells (RBCs). To account for biologic variations, cells from 10 mice that underwent MCAO in the same surgery session were pooled for each sample: (1) MCAO and (2) MCAO+PD-L1. The cells were then washed with PBS and stained with CD3, CD45, CD11b, and PD-1 (Table 3). Dead cells were excluded with propidium iodide (PI) staining. PD-1+ versus PD-1− monocytes (CD45+CD11b+CD3−) were gated and sorted into sterile RPMI (Gibco) supplemented with 5% FBS (Fisher). Methods for Single-Cell RNA-Sequencing using the 10× Genomics Platform are described herein.

TABLE 3
Antibodies used for flow cytometry of murine tissue samples.

| Antibody (Flow) | Clone | Fluorophore | Dilution | Company |
|---|---|---|---|---|
| CD3 | 145-2C11 | PercpCy5.5 | 5:200 | Biolegend |
| CD45 | 30-F11 | APC-Cy7 | 1:200 | Biolegend |
| CD11b | M1/70 | AF700 | 1:200 | Biolegend |
| CD11c | N418 | APC | 1:200 | Biolegend |
| Ly6C | HK1.4 | FITC | 1:200 | Biolegend |
| Ly6G | 1A8 | BV650 | 1:200 | Biolegend |
| CD43 | S11 | PE-Cy7 | 1:200 | Biolegend |
| PD-1 | RMP1-30 | BV421 | 1:200 | Biolegend |
| CCR2 | SA203G11 | PE | 1:200 | Biolegend |

Differential gene expression, geneset enrichment analysis, clustering, and visualization of the single-cell RNA sequencing data was performed in R (version 4.1.0) as follows:

The cell-by-gene count matrix was filtered to exclude cells with fewer than 1000 total RNA counts across all genes and to exclude genes with fewer than 10 counts across all cells. The resulting cell-by-gene matrix contained 15396 genes and 27431 cells. Counts were then normalized using library size normalization and scaled by 1e6.

To test for differentially expressed genes between PD-L1 treated and untreated monocytes, cells from the PD-1 positive and negative compartments for each treatment condition were combined. Differentially expressed genes were determined using a two-sided Wilcoxon rank sum test with a Benjamini-Hochberg multiple hypothesis correction and a p-value cutoff of 0.05. Log fold changes in gene expression between PD-L1 treated and untreated monocytes were computed as the log_2 (mean gene expression in treated cells/mean gene expression in untreated cells), where gene expression is the library size normalized and scaled counts.

Gene set enrichment analysis (A. Subramanian, et al., Proc. Natl. Acad. Sci. U.S.A 102, 15545-15550 (2005)) was performed using the fgsea package (version 1.20.0) (G. Korotkevich, et al., Fast gene set enrichment analysis. (2016)). Significantly differentially expressed genes were ranked by decreasing log fold change. GSEA was performed with the Gene Ontology Biological Processes gene sets (A. Liberzon, et al., Bioinformatics. 27, 1739-1740 (2011)), with significantly enriched genesets determined using an adjusted p-value cutoff of 0.05.

To obtain cell clusters, the CPM-normalized count matrix was first log_10 transformed with a pseudocount of 1. Principal components analysis (PCA) was performed on the centered and unit scaled normalized count matrix using the svds function in the package RSpectra (version 0.16) with 50 principal components. A nearest neighbor graph with parameter k=100 was constructed using the nn2 function in the package RANN (version 2.6.1) and clusters were obtained using the cluster_louvain function in the package igraph (version 1.2.11). To test if the proportion of treated cells in each cluster was significantly different from the proportion of treated cells in the overall data, the prop.test function in the package stats in base R was used.

The data were visualized in a two-dimensional uniform manifold approximation and projection embedding constructed using the umap function in the package uwot (version 0.1.11) using the 50 principal components and parameters n_neighbors=500, and min_dist=1, with other parameters with default values.

Single Cell RNA Sequencing. Cell counts and viabilities were determined using the Cell Countess 3 with trypan blue staining. A maximum volume of 77.4 μL/sample was used for processing to target up to 10,000 cells. Cells were combined with RT reagents and loaded onto 10λ Next GEM Chip N along with 5′ HT gel beads. The NextGEM protocol was run on the 10λ Chromium X to create GEMs (gel bead in emulsion), composed of a single cell, gel bead with unique barcode and UMI primer, and RT reagents. Approximately 180 μL of emulsion was retrieved from the chip, split into 2 wells, and incubated (45 min at 53° C., 5 min at 85° C., cool to 4° C.), generating barcoded cDNA from each cell. The GEMs were broken using Recovery Agent and cDNA was cleaned, following manufacturer's instructions using MyOne SILANE beads. cDNA was amplified for 11-16 cycles (45 sec @ 98° C., X cycle: 20 sec @ 98° C., 30 sec @ 63° C., 1 min @ 72° C.; 1 min @ 72° C., cool to 4° C.). Samples were cleaned using 0.6λ SPRIselect beads. Quality control (QC) assays were completed using Qubit and Bioanalyzer to determine size and concentrations. 20 μL of amplified cDNA was carried into library preparation. Fragmentation, end repair, and A-tailing were completed (5 min @ 32° C., 30 min @ 65° C., cool to 4° C.), and samples were cleaned up using double sided size selection (0.6×, 0.8×) with SPRIselect beads. Adaptor ligation (15 min @ 20° C., cool to 4° C.), 0.8× cleanup, and amplification are performed, with PCR using unique i7 and i5 index sequences. Libraries underwent a final cleanup using double sided size selection (0.6×, 0.8×) with SPRIselect beads. Library QC was performed using Qubit, Bioanalyzer, and KAPA library quantification qPCR kit. Libraries were sequenced on the Illumina NovaSeq 6000 using v1.5 kits, targeting 50K reads/cell, at read lengths of 28 (R1), 10 (17), 10 (i5), 91 (R2). Demultiplexing and FASTQ generation was completed using Illumina's BaseSpace software.

The binary base call (bcl) sequence files from the NovaSeq6000 (Illumina) were converted to FASTQ sequence files using BCL Convert Software (Illumina; bcl2fastq2 v2.20). The FASTQs corresponding to each library from the sequencing run were aligned and count matrices were generated using the Cell Ranger count function (10× Genomics; version cellranger-7.0.0). This matrix included single-cell RNA counts from the two sorted compartments of each of the PD-L1 treated and untreated monocytes.

Statistical Analyses. Murine data. Survival was analyzed by Kaplan-Meier method and compared by log-rank Mantel Cox test. Unpaired non-parametric Mann-Whitney tests were used to make comparisons between two groups. Comparisons within groups were presented as mean±SEM. The murine data excluding the behavior tests were analyzed using GraphPad Prism 9 and values of p<0.05 were considered significant.

28 point neuroscore. The 28 point neuroscores at week 1 for treated and untreated wild-type mice were categorized by quartile (1st, 2nd, 3rd, 4th), such that the 4th quartile represented the mice scoring in the top 25th percentile on the 28 point neuroscore. Subsequently, the Chi-squared and Fisher exact tests were used to evaluate the association between treatment with PD-L1 and having a high 28 point neuroscore (falling in the 4th quartile vs falling in any one of the 1st three quartiles). The same process (categorization followed by Chi-squared and Fisher exact testing) was implemented for global PD-1 KO and then myeloid-specific PD-1 KO mice.

DigiGait and open field. Scores on the various variables of digigait at week 1 and week 3 were compared between sham, treated stroke, and untreated stroke mice using the Kruskal-Wallis test. Post-hoc comparisons with Bonferroni correction were subsequently performed for variables with significant overall Kruskal-Wallis p-values. To provide a more global picture of the effect of PD-L1 on variables that are significantly altered by stroke compared to sham, the results for these variables were plotted in the form of horizontal bar graphs delineating the percent change in median compared to sham in treated versus untreated mice. A similar process was used to analyze the open field variables at week 4.

Human patient data. Baseline characteristics of enrolled patients were summarized using descriptive statistics (n, % for categorical variables and mean±SD with median, IQR for continuous variables). Regarding flow cytometry data, the MFI values for PD-1 fluorescence on different monocyte cell populations (total, classical, intermediate, nonclassical) were plotted at different time points. Maximum values and daily average values were assessed. Since some patients had several values collected at different time-points within the first 24-hour period since time last known well, the maximum value for the first 24 hours was selected as the “day 1” value for the daily average calculations. Non-parametric analyses using Spearman's correlation coefficient were performed to evaluate correlations between the MFI values for PD-1 fluorescence on monocyte cell subpopulations and edema-to-infarct ratios on MRI. Further analyses investigating potential linear associations were conducted using Pearson's correlation coefficient while acknowledging limitations of small sample size and the presence of outliers. Statistical significance was set at p<0.05 with 2-sided hypothesis testing, and analyses were performed using SPSS software (version 25.0; SPSS Inc, IBM, Armonk, New York).

In vitro polarization of myeloid cells. Hematopoietic stem cells were isolated from the femur and tibia of C57BL/6 mice and treated with ACK lysing buffer for red blood cell lysis. Cells were plated with myeloid-polarizing cytokines-murine M-CSF (40 ng/ml) and GM-CSF (40 ng/ml), for 2 days. Cells were plated on control or PD-L1-immobilized plates (pre-coated with 1 ug/ml of soluble PD-L1 overnight) and with isotype control or anti-PD-1 (1 μg/ml or 10 μg/ml doses). On day 3, flow cytometry was conducted to assess monocyte polarization.

Seahorse assay for measurement of mitochondrial respiration. Hematopoietic stem cells were isolated from the femur and tibia of C57BL/6 mice and treated with ACK lysing buffer for red blood cell lysis. Cells were plated with myeloid-polarizing cytokines, murine M-CSF (40 ng/ml) and GM-CSF (40 ng/ml), for 2 days. Cells were plated on control or PD-L1-immobilized plates (pre-coated with 1 μg/ml of soluble PD-L1 overnight) and with isotype control or anti-PD-1 (1 μg/ml or 10 μg/ml doses). On the third day, polarization to myeloid cells was confirmed by flow cytometry. Mitostress test was performed with the polarized cells using the Seahorse XF Pro Analyzer (Agilent Technologies, Massachusetts, USA). Prior to seeding on Seahorse cell culture microplates, myeloid cells were washed with buffered Seahorse XF Base Medium. After washing the cells, 200,000 cells were transferred to the microplates and incubated with buffered Seahorse XF Base Medium supplemented with 2 mM glutamate, 1 mM sodium pyruvate and 10 mM glucose for an hour at 37° C. in a non-CO2 incubator. Oxygen consumption rate (OCR) was measured under basal conditions and in response to 1.5 μM Oligomycin, 1 μM fluoro-carbonyl cyanide phenylhydrazone (FCCP), and 100 nM rotenone+1 μM antimycin A.

Gait Analysis. The DigiGait Imaging System (Mouse Specifics, Boston, MA) was used for gait capture and analysis. The mouse-specific camera and treadmill enclosure were used. Before each run, the clarity of the image capture was optimized for paw capture. The paws were not painted or altered. Customized overhead white LED strip lights were placed on the top of the mouse treadmill enclosure to increase the contrast between the background and the paws. A speed of 10 cm/s was used at 1-week post-ischemia and 15 cm/s at 3-week post-ischemia. These speeds were determined using training cohorts of post-ischemic mice. The highest speed possible was used such that at least 75% of mice at each time point could adequately maintain gait at that speed for at least 10 steps. Before official image capture, mice were allowed at least one training run on the treadmill to acclimate to the speed and task. Mice were allowed up to three attempts over two trials on the day of recording to maintain consistent gait for at least six strides (six steps with each paw). The quality of the paw capture and step tracings were evaluated in real time and adjustments were made to noise filters and other parameters before being satisfied with the recording. At least three segments of at least 6-10 steps (without stumbling, contacting a wall or bumper, or turning) each were recorded before capture was completed. Mice who could not satisfy these requirements were excluded from that time point.

For the analysis portion, for each mouse the entire captured video was reviewed. The highest quality segment with at least 6-10 steps was selected. Noise filtering was performed in the Digigait software. Once paw tracings were produced, the actual recording was rewatched and correlated to the tracings to ensure noise was not captured as a step (e.g., the tail or genitalia were not counted as paw) and that steps were not missed. This was corrected manually if possible, including truncating that step if needed. Care was taken not to “over-filter” the tracings such that potential differences in gait pattern were not eliminated. If there were errors that could not be corrected, an alternate segment of video capture was trialed. If the same issue arose, that mouse was excluded. As a quality assessment, the subjective quality of the final tracings was reviewed, as well as calculated parameters, such as mouse length. The indices calculated by the software for good quality mice were then exported for analysis. Indices definitions are provided in prior literature (J. Vincelette, et al. Arthritis Res Ther. 9, R123 (2007) 61, 65); and E. R. Berryman, et al. J Musculoskelet Neuronal Interact. 9, 89-98 (2009))

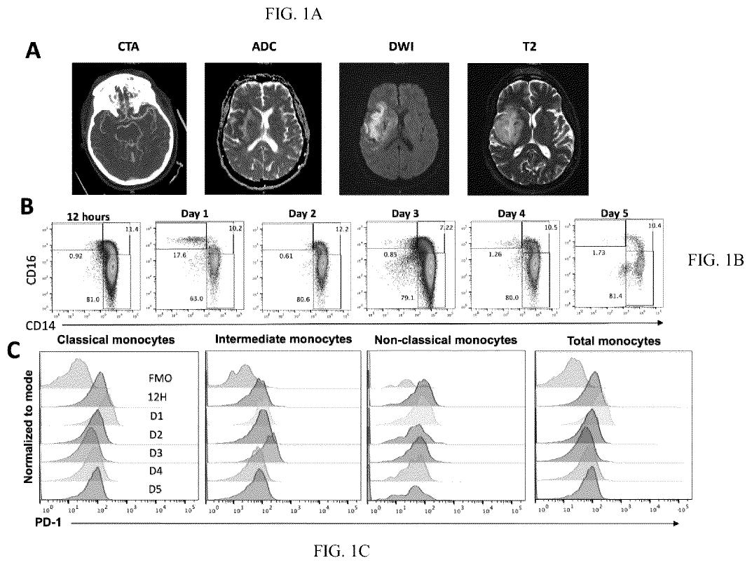

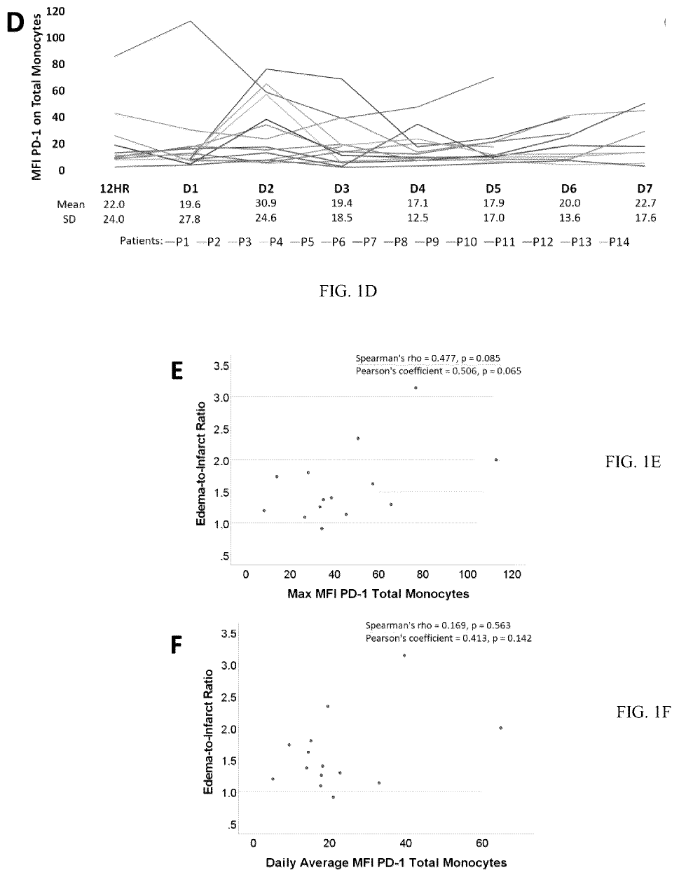

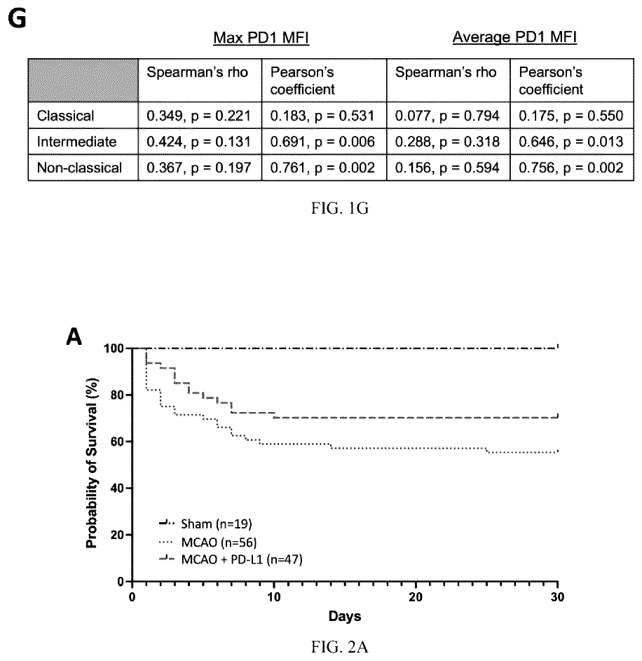

Results. PD-1 was expressed on monocytes and correlated with radiographic edema in LVO patients. To determine if circulating PD-1+ monocytes correlate with neurologic outcomes in humans, 14 patients who presented with an LVO of the right or left MCA were studied. CT angiography (CTA) showing a right MCA occlusion and MRI sequences showing stroke volume (ADC and DWI) and surrounding edema (T2) from a representative case are presented in FIG. 1A. The average age was 62 years, and the most frequently affected vessel was the M1 segment (most proximal) of the left MCA. Baseline and imaging outcome characteristics are presented in Table 4. A total of 12 patients (86%) underwent mechanical thrombectomy, and the mean edema-to-infarct ratio on magnetic resonance imaging (MRI) was 1.6. PD-1 expression was observed in classical (CD14hi, CD16lo), intermediate (CD14hi, CD16hi), and non-classical (CD14lo, CD16lo) monocyte populations (FIG. 1B need to add breakdown of PD-1+ cells in each subgroup for FIG. 6). The pattern of PD-1 staining on monocytes was a shift rather than the well-delineated PD-1+ population observed on lymphocytes. This pattern is consistent with previous reports of PD-1 staining on myeloid cells (25, 39, 40). The highest PD-1 fluorescence on total monocytes was observed on day 2 (30.9±24.6); however, there was considerable inter- and intra-patient variability in timing and degree of PD-1 expression on monocytes as demonstrated by the individual patient graphs in FIG. 1C. Higher daily average (FIG. 1D) and maximum PD-1 MFI values on total monocytes (FIG. 1E) correlated with increased edema-to-infarct ratios graphically, with trends towards significance noted for max values (Spearman's rho=0.477, p=0.085, Pearson's correlation coefficient=0.506, p=0.065). Significant correlations between MFI for PD-1 and edema-to-infarct ratio were noted in our Pearson correlation analysis in the intermediate and nonclassical monocyte subtypes.

TABLE 4
Patient demographics and clinical characteristics.

| | |
|---|---|
| | No. (%) or Mean ± SD, |
| Characteristic | median (IQR) |

| | | |
|---|---|---|
| Sex, female | 9 | (64) |

| | |
|---|---|
| Age, yrs | 61.6 ± 17.4, 65.5 (24.5) |
| Large vessel occlusion | |

| | | |
|---|---|---|
| Left MCA | 9 | (64) |
| Right MCA | 5 | (36) |

ASPECTS on arrival (n = 13)*

| | | |
|---|---|---|
| 5 | 1 | (7) |
| 6 | 1 | (7) |
| 7 | 1 | (7) |
| 8 | 3 | (21) |
| 9 | 4 | (29) |
| 10 | 3 | (21) |

| | |
|---|---|
| NIHSS score on arrival | 16.9 ± 5.0, 18.0 (8.5) |

| | | |
|---|---|---|
| tPA administered | 9 | (64) |

| | |
|---|---|
| Time from last known well | 6.8 ± 5.6, 4.7 (6.5) |
| to presentation, hrs | |

| | | |
|---|---|---|
| Mechanical thrombectomy | 12 | (86) |
| performed | | |

| | |
|---|---|
| Time from last known well | 8.8 ± 6.3, 7.5 (8.1) |
| to recanalization, hrs | |
| TICI score | |

| | | |
|---|---|---|
| 2a | 1 | (8) |
| 2b | 5 | (42) |
| 2c | 2 | (17) |
| 3 | 4 | (33) |
| Stent placed | 1 | (8) |

| | |
|---|---|
| Edema volume on MRI, cm³ | 84.4 ± 62.3, 72.9 (103.3) |
| Infarct volume on MRI, cm³ | 61.5 ± 48.4, 58.1 (100.8) |
| Edema-to-infarct ratio | 1.6 ± 0.6, 1.4 (0.7) |

ASPECTS: Alberta stroke program early CT score;
ICA: internal carotid artery;
NIHSS: National Institutes of Health Stroke Scale;
TICI: thrombolysis in cerebral infarction;
tPA: tissue plasminogen activator
*Number in parentheses represents patients without missing data

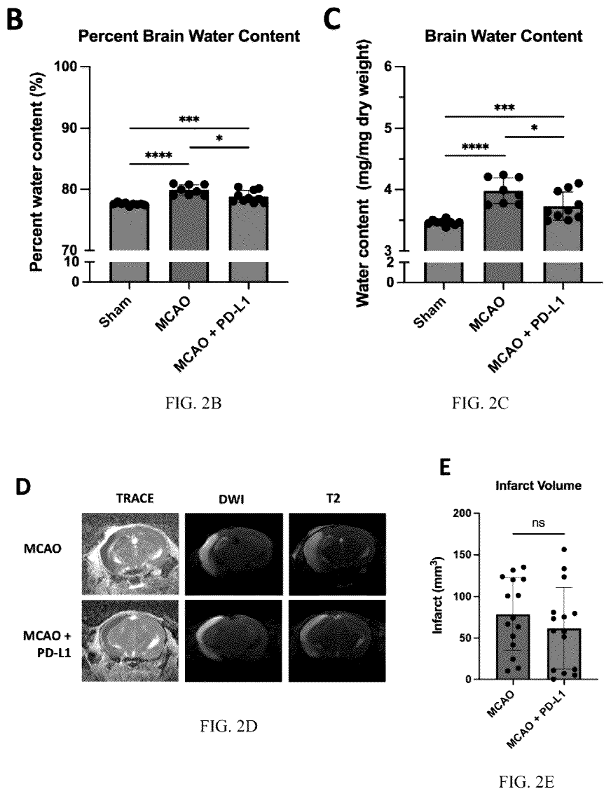

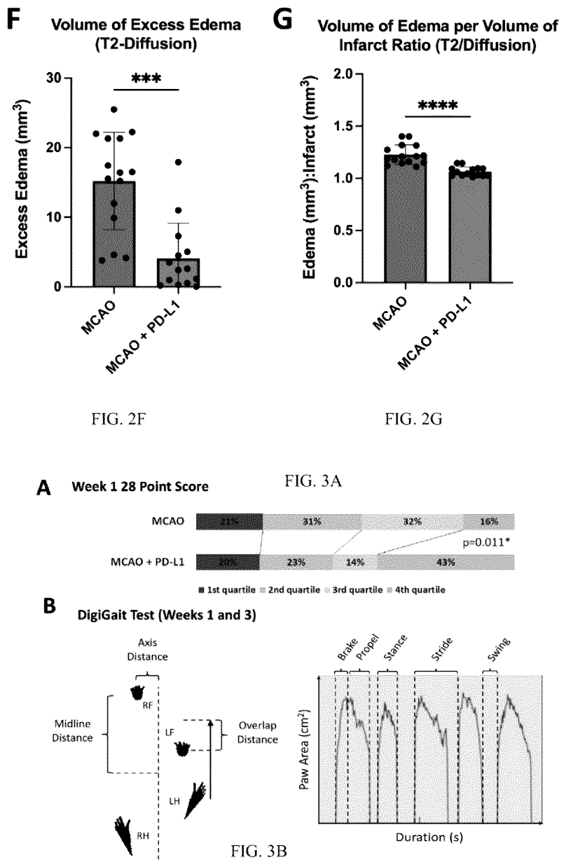

Administration of soluble PD-L1 reduced cerebral edema and improved survival in mice. The effects of IP injection of sPD-L1 (administered at 1 hour and 24 hours after ischemic insult) was evaluated on functional and radiographic outcomes in a transient MCAO model. Administration of sPD-L1 improved overall survival, particularly in the first 48 hours after infarct (FIG. 2A). The overall survival rate for the untreated MCAO mice was 55.36% (n=56), versus 70.21% for MCAO mice that received sPD-L1 (n=47; χ2=12.23, p=0.0022).

Because the difference in survival was most evident in the acute phase, it was tested whether treatment with sPD-L1 decreased early brain swelling. Brain water content was measured at 48 hours post-MCAO (FIG. 2B,C) (R. F. Keep, et al., A misunderstood measurement? Transl Stroke Res. 3, 263-265 (2012)). Compared to animals that underwent sham surgery, mice undergoing MCAO without treatment had a 2.28% increase in whole brain water content (77.61% and 79.89%, respectively, p=<0.0001).

Treatment with sPD-L1 significantly decreased the percent water content to 78.82% (p=0.0266). To quantify the relationship between infarct and edema volumes, high resolution T2, DWI, and ADC MRI sequences were obtained at 72 hours post-MCAO (FIG. 2D). Total infarct volume, as visualized by areas of restricted diffusion on both DWI and ADC maps, was not significantly changed by sPD-L1 treatment (FIG. 2E). However, treated mice had significantly lower volumes of excess edema, as defined by total volume of T2 hyperintensity subtracted by the total volume of diffusion restriction (p<0.0001) (FIG. 2F). This relationship remained significant when normalizing for infarct volume (p<0.0001) (FIG. 2G).

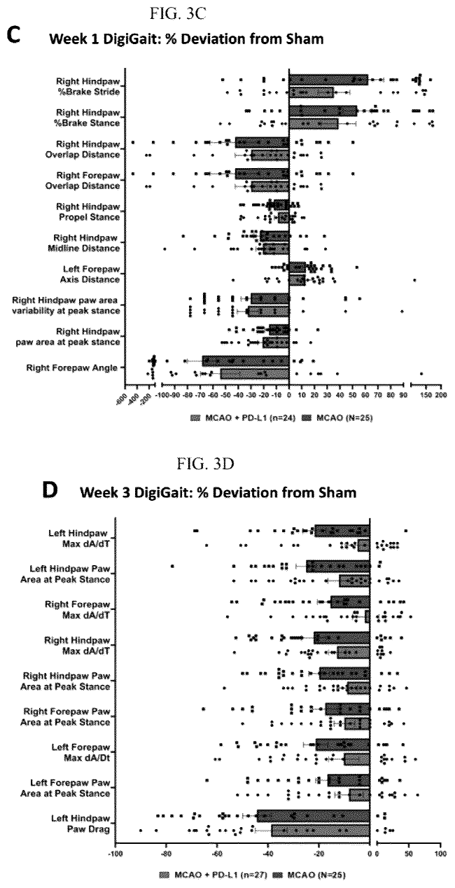

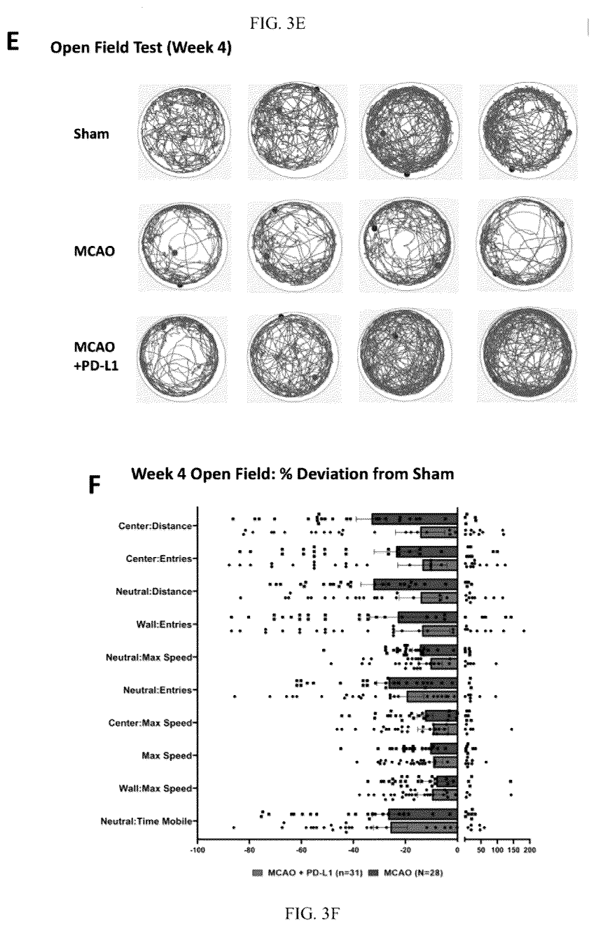

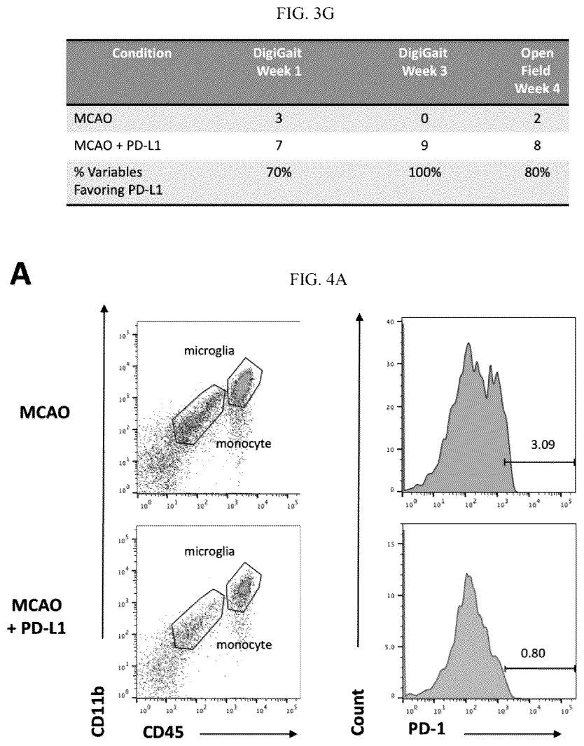

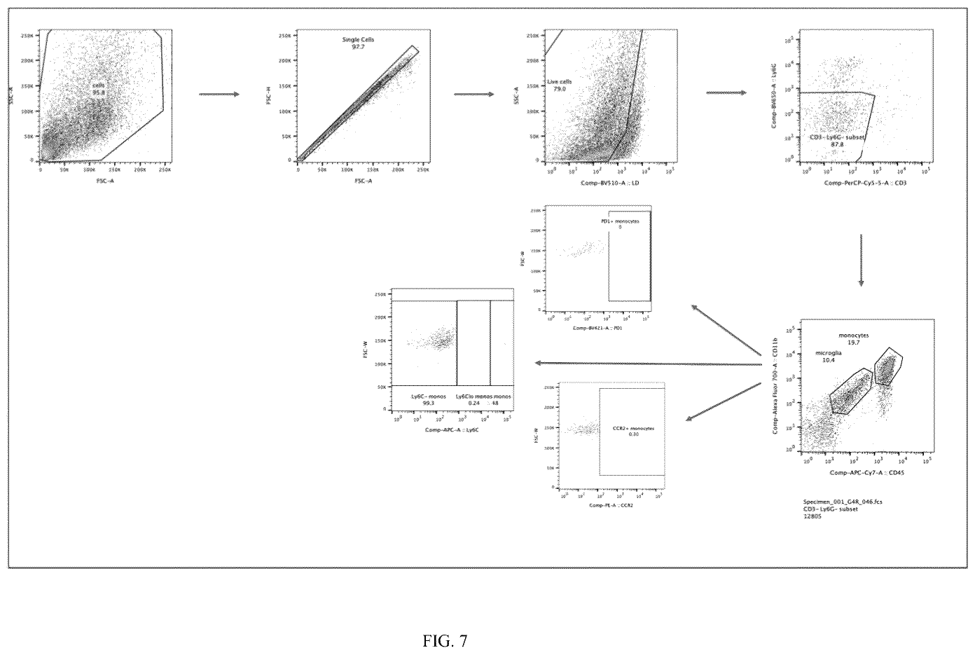

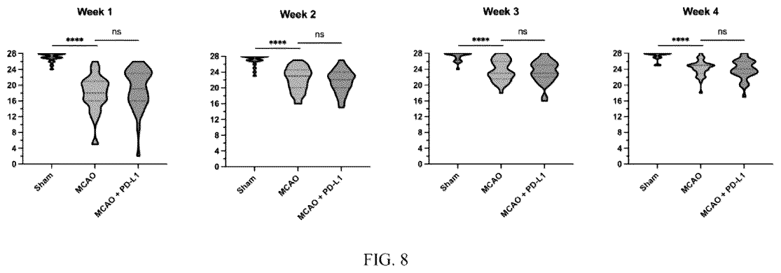

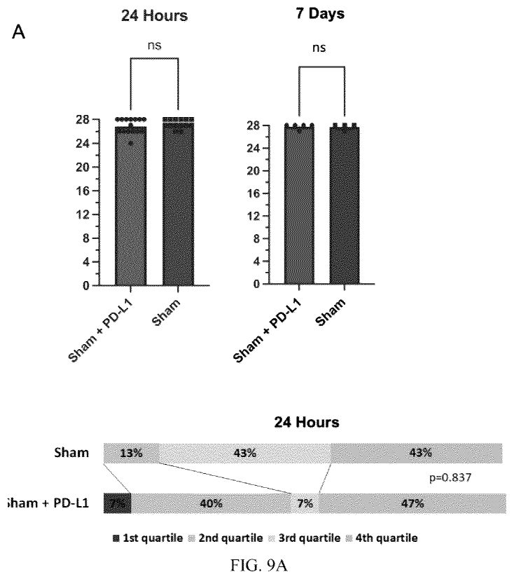

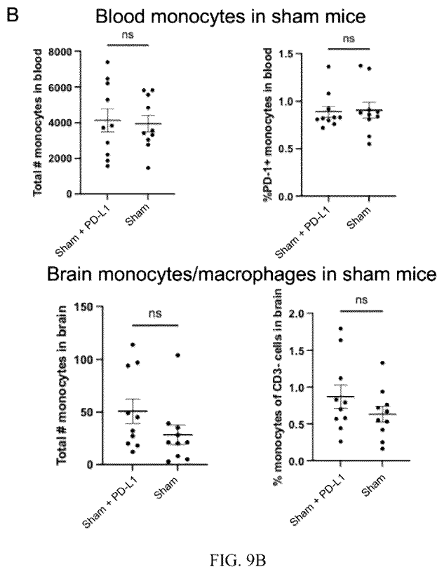

Soluble PD-L1 improved early performance scores in mice with potential for good functional recovery, but did not improve early recovery in severely injured mice. To evaluate functional outcomes, the 28-point neuroscore—a well-established sensorimotor behavior test (A. Encarnacion, et al., J Neurosci Methods. 196, 247-257 (2011))—was administered weekly after MCAO. Like humans, mice have a range of outcomes after stroke. To account for this variation the scores were broken down into quartiles to determine if subgroups of mice were affected by treatment (i.e., categorized by 1st, 2nd, 3rd and 4th quartiles such that the 4th quartile represented the mice scoring in the top 25th percentile on the 28 point neuroscore). Animals treated with sPD-L1 were significantly more likely to fall into the highest quartile (71%, p=0.011) (FIG. 3A). Based on raw scores alone, there was no significant difference between the untreated and treated cohorts (FIG. 8). By weeks 2, 3, and 4, a difference was no longer discernable (Tables 1, 5, and 6), consistent with previous reports of good spontaneous recovery after MCAO as measured by 28-point neuroscore (A. Encarnacion, et al., J Neurosci Methods. 196, 247-257 (2011)). These findings indicate that mice with a higher potential for functional recovery benefit from sPD-L1, while the effects of immune activation are overshadowed by the primary ischemic injury in poorly performing mice. sPD-L1 in sham operated mice did not affect 28-point neuroscore or immune phenotype of circulating or brain infiltrating myeloid cells (FIG. 9).

TABLE 5
28-point score quartile breakdown in MCAO vs. MCAO +
PD-L1 PD-1 knockout mice, with P-value
comparisons for falling in the highest quartile.

| | 1 | 2 | 3 | 4 | P-value |
|---|---|---|---|---|---|
| | Week 2 Quartiles | | | | |
| MCAO | 3 (13) | 6 (26) | 4 (17) | 10 (43) | 0.618 |
| MCAO + PD-L1 | 7 (26) | 6 (22) | 5 (19) | 9 (33) | |
| | Week 3 | | | | |
| MCAO | 5 (22) | 5 (22) | 6 (26) | 7 (30) | 0.488 |
| MCAO + PD-L1 | 5 (19) | 8 (31) | 3 (12) | 10 (38) | |
| | Week 4 | | | | |
| MCAO | 4 (17) | 5 (22) | 5 (22) | 9 (39) | 0.311 |
| MCAO + PD-L1 | 5 (21) | 7 (29) | 6 (25) | 6 (25) | |

TABLE 6
28-point score quartile breakdown in MCAO vs. MCAO +
PD-L1 PD-1 myeloid-specific knockout mice, with P-value
comparisons for falling in the highest quartile.

| | 1 | 2 | 3 | 4 | P-value |
|---|---|---|---|---|---|
| | Week 2 Quartiles | | | | |
| MCAO | 1 (20) | 2 (40) | 2 (40) | 0 (0) | 0.592 |
| MCAO + PD-L1 | 1 (14) | 3 (43) | 1 (14) | 2 (29) | |
| | Week 3 | | | | |
| MCAO | 0 (0) | 1 (50) | 1 (50) | 0 (0) | 0.999 |
| MCAO + PD-L1 | 1 (25) | 1 (25) | 1 (25) | 1 (25) | |
| | Week 4 | | | | |
| MCAO | 0 (0) | 0 (0) | 2 (100) | 0 (0) | 0.999 |
| MCAO + PD-L1 | 1 (25) | 1 (25) | 1 (25) | 1 (25) | |

PD-L1 treated mice more closely resembled sham operated animals on assessments of long-term functional recovery. To assess long-term functional outcomes, locomotor function was assessed using the Digigait system (FIG. 3B). Significance was determined by a p value <0.05 after a Bonferroni correction for multiple comparisons (Table 7, 8). At 1-week post-MCAO, computerized treadmill gait analysis of the sham surgery versus the untreated control cohorts revealed 10 gait parameters that were significantly altered by stroke. In the sPD-L1 treatment cohort, 7 of these 10 parameters were closer to sham than the untreated stroke group (FIG. 3C). At week three, the parameters (9/9) that were significantly affected by stroke showed a trend towards improvement with PD-L1 treatment (FIG. 3D). The open field test was administered at week four to evaluate overall activity levels, locomotor ability, exploration habits, and anxiety (FIG. 3E). Comparison of the sham versus untreated animals revealed ten parameters that were significantly altered by stroke. Eight of these variables trended closer to sham animals in the PD-L1 treated group (FIG. 3F). Due to high variance in these interconnected behavioral measures, none of the individual variables reached significance for treated vs. untreated stroke mice (Tables 7, 8, 9).

TABLE 7
Digigait test results at week 1 after MCAO. Listed by paw are the variables that showed both significance between the three groups on the overall
Kruskal Wallis test and a significant difference between the sham group and the untreated stroke group. Therefore, the listed variables are the
gait parameters affected by stroke. Significance was determined by a p value < 0.05 after a Bonferroni correction for multiple comparisons.

| | | | Sham vs | | Sham vs | Untreated |
|---|---|---|---|---|---|---|
| Overall | Sham | Untreated Stroke | Untreated | Treated Stroke | Treated | vs Treated |
| Significance | (median, IQR) | (median, IQR) | Stroke | (median, IQR) | Stroke | Stroke |

LEFT FORE
PAW

| Axis Distance | p = 0.018* | −0.75 | (−0.83, −0.66) | −0.88 | (−0.91, −0.74) | p = 0.021* | −0.84 | (−0.93, −0.72) | p = 0.063 | p = 0.999 |
|---|---|---|---|---|---|---|---|---|---|---|
| RIGHT | | | | | | | | | | |
| FORE PAW | | | | | | | | | | |
| Paw Angle | p = 0.011* | −0.1 | (−4.3, 4.2) | 6.3 | (1.9, 11.4) | p = 0.010* | 6.3 | (0.5, 9.2) | p = 0.055 | p = 0.999 |
| Overlap | p = 0.026* | 2.4 | (2.2, 3.0) | 1.8 | (1.1, 2.5) | p = 0.029* | 2.1 | (1.6, 2.5) | p = 0.085 | p = 0.999 |
| Distance | | | | | | | | | | |
| LEFT HIND | | | | | | | | | | |
| PAW | | | | | | | | | | |
| — | — | — | | — | | — | — | | — | — |
| RIGHT | | | | | | | | | | |
| HIND PAW | | | | | | | | | | |
| % BrakeStride | p = 0.041* | 11.1 | (10.3, 13.8) | 19.1 | (12.2, 24.0) | p = 0.040* | 12.5 | (9.7, 20.4) | p = 0.083 | p = 0.830 |
| % BrakeStance | p = 0.041* | 18.1 | (14.8, 23.5) | 28.8 | (19.8, 32.2) | p = 0.034* | 21.6 | (15.7, 32.1) | p = 0.375 | p = 0.815 |
| % PropelStance | p = 0.041* | 82 | (77-85) | 71 | (68, 80) | p = 0.034* | 78 | (68, 84) | p = 0.375 | p = 0.815 |
| Overlap | p = 0.026* | 2.39 | (2.15, 2.96) | 1.82 | (1.09, 2.53) | p = 0.029* | 2.10 | (1.60, 2.49) | p = 0.085 | p = 0.999 |
| Distance | | | | | | | | | | |
| Paw Area at | p = 0.004* | 0.53 | (0.48, 0.56) | 0.45 | (0.40, 0.51) | p = 0.040* | 0.44 | (0.36, 0.48) | p = 0.004* | p = 0.999 |
| Peak Stance | | | | | | | | | | |
| Paw Area | p = 0.014* | 0.09 | (0.07, 0.11) | 0.05 | (0.03, 0.08) | p = 0.038* | 0.05 | (0.04, 0.08) | p = 0.021* | p = 0.999 |
| Variability at | | | | | | | | | | |
| Peak Stance | | | | | | | | | | |
| Midline | p = 0.002* | 2.34 | (2.11, 2.47) | 1.74 | (1.56, 2.06) | p = 0.002* | 1.91 | (1.72, 2.15) | p = 0.020* | p = 0.999 |

Distance

TABLE 8
DigiGait test results at week 3 after MCAO. Listed by paw are the variables that showed both significance between the three groups on the overall
Kruskal Wallis test and a significant difference between the sham group and the untreated stroke group. Therefore, the listed variables are the
gait parameters affected by stroke. Significance was determined by a p value < 0.05 after a Bonferroni correction for multiple comparisons.

| | | | Sham vs | | Sham vs | Untreated |
|---|---|---|---|---|---|---|
| Overall | Sham | Untreated Stroke | Untreated | Treated Stroke | Treated | vs Treated |
| Sig | (median, IQR) | (median, IQR) | Stroke | (median, IQR) | Stroke | Stroke |

LEFT FORE
PAW

| | | | | | | | | | | |
|---|---|---|---|---|---|---|---|---|---|---|
| Paw Area at | p = 0.029* | 0.25 | (0.24, 0.28) | 0.21 | (0.17, 0.24) | p = 0.025* | 0.22 | (0.17, 0.27) | p = 0.215 | p = 0.999 |
| Peak Stance | | | | | | | | | | |
| Max dA/dT | p = 0.008* | 19.9 | (17.8, 24.8) | 15.5 | (11.9, 18.2) | p = 0.006* | 16.5 | (13.2, 22.2) | p = 0.143 | p = 0.632 |
| RIGHT | | | | | | | | | | |
| FORE PAW | | | | | | | | | | |
| Paw Area at | p = 0.034* | 0.26 | (0.23, 0.28) | 0.21 | (0.18, 0.25) | p = 0.029* | 0.23 | (0.20, 0.26) | p = 0.263 | p = 0.978 |
| Peak Stance | | | | | | | | | | |
| Max dA/dT | p = 0.013* | 19.9 | (17.2, 26.3) | 16.2 | (13.3, 18.2 | p = 0.014* | 18.8 | (16.3, 22.5) | p = 1.000 | p = 0.999 |
| LEFT HIND | | | | | | | | | | |
| PAW | | | | | | | | | | |
| Paw Area at | p = 0.002* | 0.58 | (0.52, 0.61) | 0.45 | (0.36, 0.52) | p = 0.001* | 0.54 | (0.41, 0.58) | p = 0.216 | p = 0.149 |
| Peak Stance | | | | | | | | | | |
| Max dA/dT | p = 0.001* | 59.0 | (54.4, 67.7) | 45.7 | (41.1, 54.5) | p = 0.002* | 55.6 | (49.8, 68.1) | p = 1.000 | p = 0.999 |
| Paw Drag | p = 0.001* | −5.47 | (−6.72, −4.02) | −2.62 | (−3.85, −1.81) | p = 0.001* | −2.71 | (−4.82, −2.00) | p = 0.008* | p = 0.999 |
| RIGHT | | | | | | | | | | |
| HIND PAW | | | | | | | | | | |
| Paw Area at | p = 0.001* | 0.56 | (0.52, 0.61) | 0.43 | (0.36, 0.52) | p = 0.001* | 0.50 | (0.45, 0.56) | p = 0.136 | p = 0.171 |
| Peak Stance | | | | | | | | | | |
| Max dA/dT | p = 0.006* | 60.7 | (49.5, 69.5) | 43.1 | (37.3, 53.2) | p = 0.005* | 51.6 | (46.0, 63.3) | p = 0.428 | p = 0.192 |

TABLE 9
Open field test results at week 4 after MCAO. Listed are the variables that showed both significance between the three
groups on the overall Kruskal Wallis test and a significant difference between the sham group and the untreated stroke
group. Therefore, the listed variables are the open field parameters affected by stroke. Reversal of stroke effect
with treatment meant that the variable was significantly different between sham mice and untreated stroke mice, but
for the treated stroke mice the median returned closer to the sham group median and was no longer significantly different.
Significance was determined by a p value < 0.05 after a Bonferroni correction for multiple comparisons.

| | | | Untreated | | Treated | | Untreated |
|---|---|---|---|---|---|---|---|
| | | Sham | Stroke | Sham vs | Stroke | Sham vs | vs |
| | Overall | (median, | (median, | Untreated | (median, | Treated | Treated |
| | Sig | IQR) | IQR) | Stroke | IQR) | Stroke | Stroke |
| Center: entries | p = 0.024* | 24.5 | 18.0 | p = 0.020* | 22.0 | p = 0.157 | p = 0.999 |
| | | (20.0, 33.0) | (11.0, 24.5) | | (11.0, 30.0) | | |
| Center: distance | p = 0.008* | 2.2 | 1.4 | p = 0.006* | 1.8 | p = 0.138 | p = 0.559 |
| | | (1.7, 2.9) | (1.0, 1.9) | | (1.0, 2.6) | | |
| Neutral: distance | p = 0.001* | 10.8 | 6.9 | p = 0.001* | 8.3 | p = 0.063 | p = 0.332 |
| | | (9.0, 13.4) | (5.4, 9.0) | | (5.7, 12.2) | | |
| Wall: entries | p = 0.044* | 30.5 | 19.5 | p = 0.038* | 23.0 | p = 0.271 | p = 0.999 |
| | | (20.0, 38.0) | (12.0, 28.5) | | (14.0, 32.0) | | |
| Max speed | p = 0.005* | 0.37 | 0.31 | p = 0.012* | 0.32 | p = 0.009* | p = 0.999 |
| | | (0.35, 0.41) | (0.29, 0.37) | | (0.29, 0.38) | | |
| Center: max speed | p = 0.006* | 0.38 | 0.33 | p = 0.023* | 0.33 | p = 0.007* | p = 0.999 |
| | | (0.37, 0.41) | (0.29, 0.38) | | (0.28, 0.36) | | |
| Neutral: entries | p < 0.001* | 125.5 | 91.5 | p = 0.001* | 95.0 | p = 0.009* | p = 0.999 |
| | | (111.0, 146.0) | (74.5, 110.5) | | (72.0, 124.0) | | |
| Neutral: max speed | p < 0.001* | 0.42 | 0.34 | p < 0.001* | 0.36 | p = 0.002* | p = 0.999 |
| | | (0.40, 0.46) | (0.33, 0.38) | | (0.34, 0.41) | | |
| Neutral: time mobile | p = 0.005* | 140.9 | 98.5 | p = 0.015* | 83.8 | p = 0.008* | p = 0.999 |
| | | (127.4, 159.1) | (63.0, 140.6) | | (68.6, 137.2) | | |
| Wall: max speed | p < 0.001* | 0.43 | 0.37 | p = 0.003* | 0.36 | p = 0.001* | p = 0.999 |
| | | (0.41, 0.47) | (0.33, 0.40) | | (0.33, 0.41) | | |

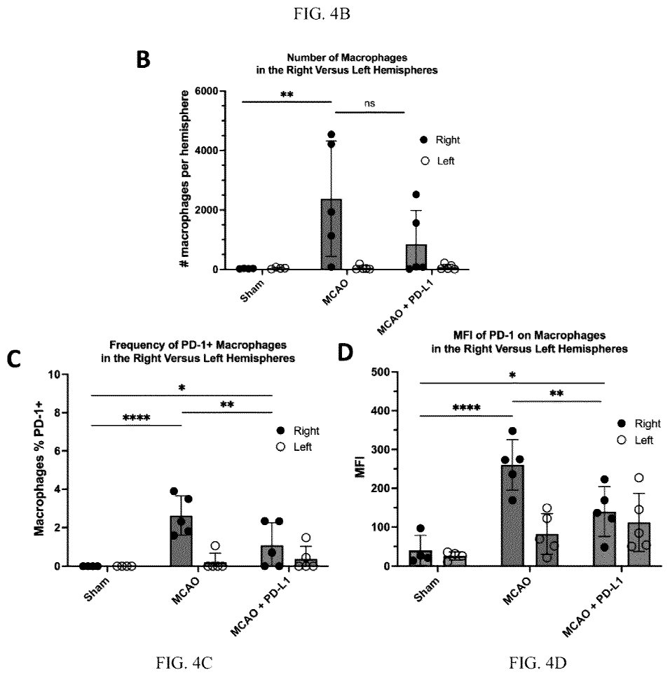

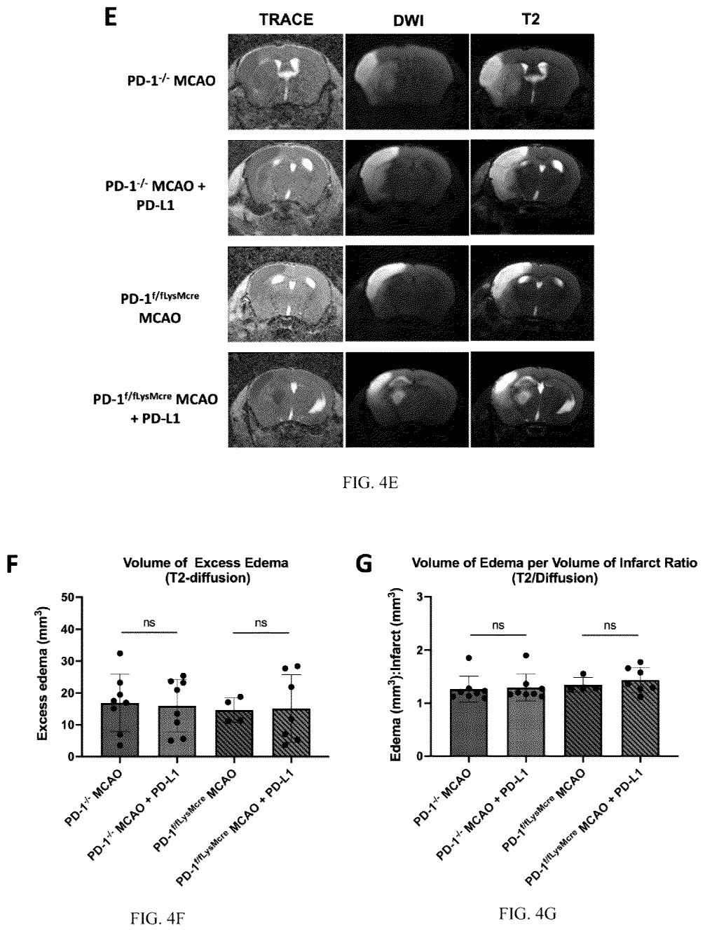

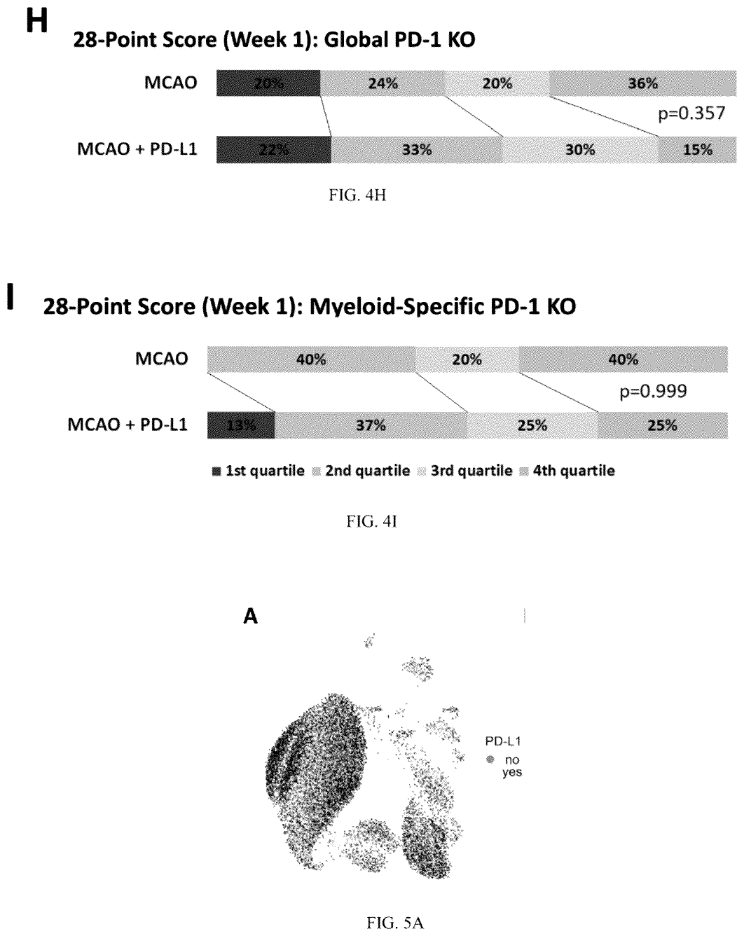

PD-1 is upregulated on infiltrating brain myeloid cells. To determine whether PD-1+ infiltrating monocytes were present in the ischemic hemisphere after stroke, immune cells were isolated from the left and right hemispheres 48 hours after MCAO (FIG. 4A). Comparison of the right versus left hemispheres revealed a significantly higher number of CD11b+, CD45 hi monocytes in the ischemic hemisphere compared to the non-ischemic hemisphere (FIG. 4B). sPD-L1 treatment resulted in a lower overall frequency of PD-1 positivity amongst the infiltrating macrophage population (p=0.0071) (FIG. 4C) by one-way ANOVA and Holm-Sidak's tests for multiple comparisons, and a trend towards decreased PD-1 expression as measured by MFI (p=0.0059) (FIG. 4D), but did not decrease the overall number of monocytes. There was no significant change in CCR2 expression on infiltrating monocytes.

Myeloid-specific knockout of PD-1 abrogated the PD-L1 treatment effect. To determine if PD-1+ myeloid cells were the targets of sPD-L1, MCAO was performed on two sets of age-matched transgenic mice: (1) global PD-1 knockout (PD-1−/−) and (2) myeloid-specific PD-1 knockout mice (PD-1f/fLysMcre). High-resolution MRI was performed at 72 hours to determine if loss of PD-1 expression would abrogate the treatment effect (FIG. 4E). Volumetric analysis revealed that sPD-L1 treatment had no effect on total infarct volume, excess edema volume, or excess edema per infarct volume in both the PD-1−/− and PD-1f/fLysMcre mice (FIGS. 4F, G). The 28-point neuroscore at week 1 was not affected by sPD-L1 administration in either the PD-1−/− or PD-If/fLysMcre mice (FIGS. 4H, I). By both metrics, the knockout mice resembled untreated mice, indicating worse outcomes after MCAO when PD-1 was ablated in myeloid cells, either in a global or tissue-specific manner.

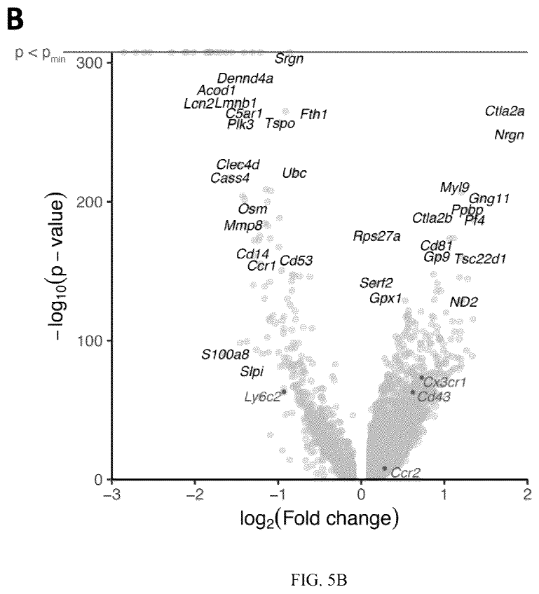

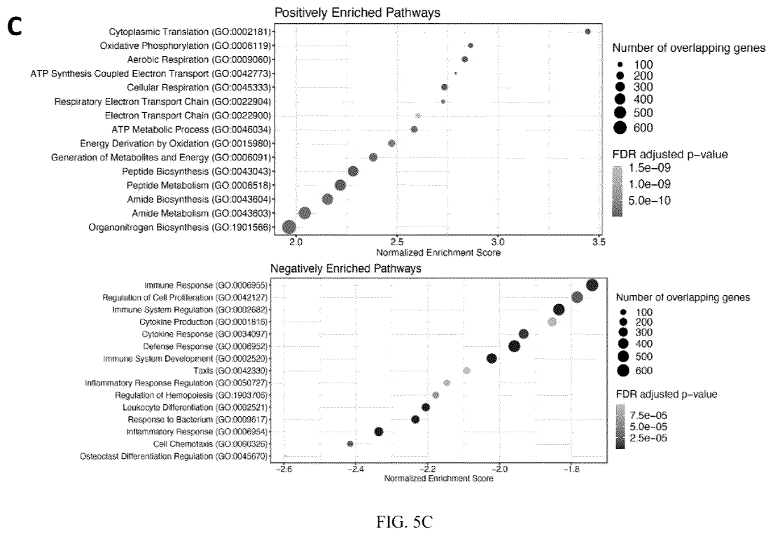

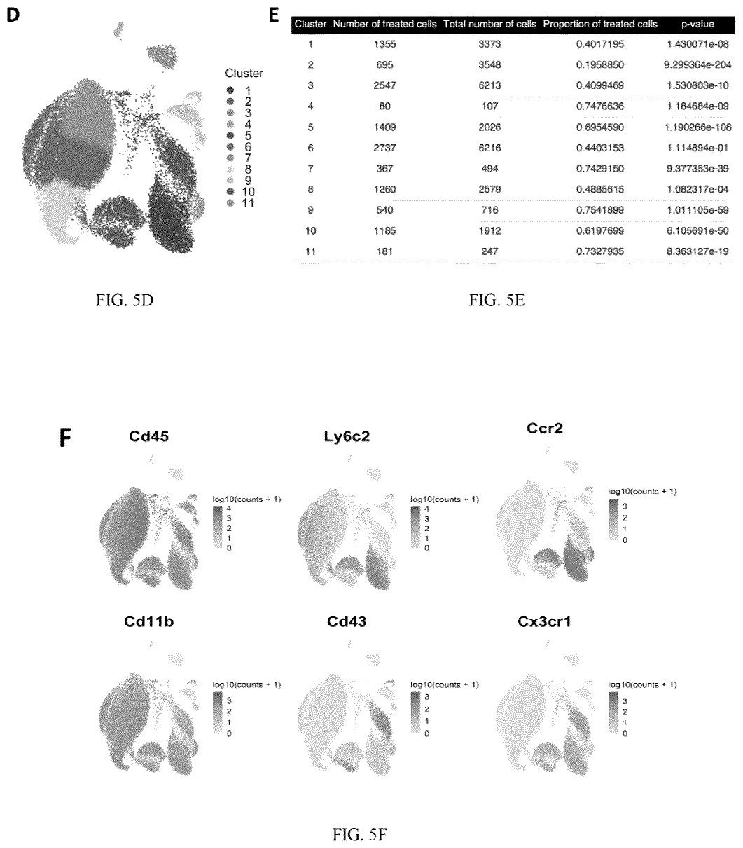

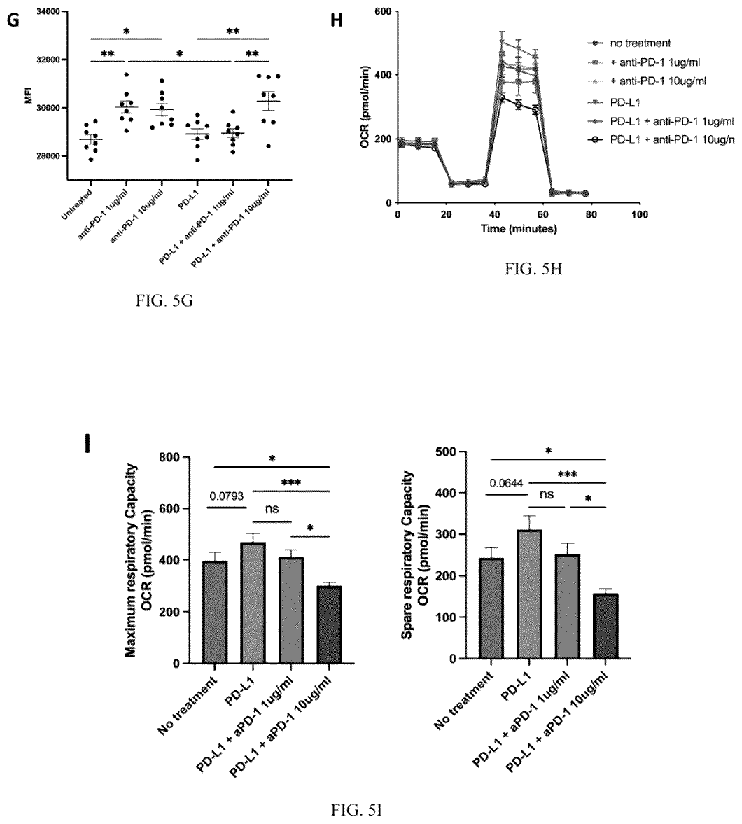

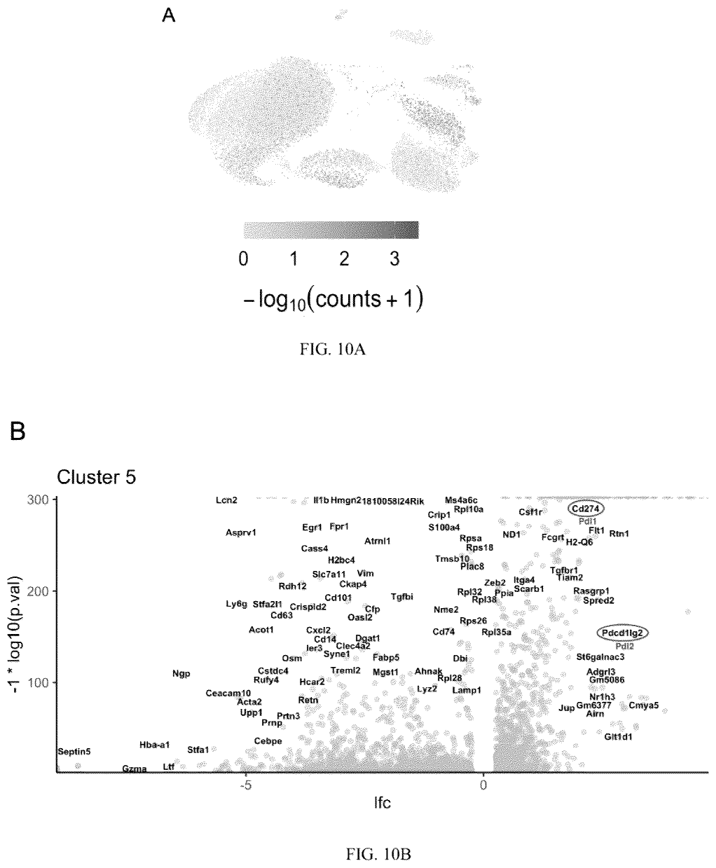

Soluble PD-L1 treatment reprograms circulating monocytes. Single cell RNA sequencing of the CD45+CD11b+CD3− immune cells in the blood 48 hours after MCAO was performed to characterize circulating monocytes in untreated and sPD-L1 treated mice (FIG. 5A). Differential gene expression analysis performed between the untreated and treated monocytes identified 5,455 differentially expressed genes (DEGs) (FIG. 5B). Gene set enrichment analysis revealed a positive enrichment in metabolism and biosynthesis genesets and a negative enrichment in immune and inflammatory process genesets in monocytes isolated from PD-L1 treated mice (FIG. 5C). From the sequenced cells, 11 clusters were identified (FIG. 5D) and proportion of cells that were derived from sPD-L1 treated mice were compared for each cluster (FIG. 5E). RNA expression of markers associated with monocyte ontogeny and activation status were evaluated to explore potential phenotypes of the monocyte subpopulations (FIG. 5F). To further investigate transcriptional differences between the monocyte subpopulations, differential gene expression between cells in each cluster and the other cells in the data was performed. Most clusters showed robust expression of CD45 and CD11b, indicating limited contamination of other cell types during the sorting process. Clusters 5 and 10, which exhibited lower expression of Ly6c2 and higher expression of Cd43, were significantly enriched in sPD-L1 treated mice. Monocyte clusters 1 and 10 displayed higher expression of Ly6c and Ccr2 (associated with a “classical” pro-inflammatory phenotype) (K. Rahman, et al. J Clin Invest. 127, 2904-2915 (2017); and M. D. Hammond, et al., J Neurosci. 34, 3901-3909 (2014)) and lower expression of Cd43, whereas monocyte cluster 5 had higher expression of Cd43 and lower expression of Ly6c and Ccr2, indicative of a “non-classical” patrolling phenotype (G. Thomas, et al., Arterioscler Thromb Vasc Biol. 35, 1306-1316 (2015)) (FIG. 5F). Of note, cluster 5 was also enriched for PD-L1+ monocytes (FIG. 10B), which has been reported to be a specific marker of non-classical monocytes (M. Bianchini, et al., Sci Immunol. 4, caar3054 (2019)). Expression of Cx3crl, a chemokine receptor that mediates anti-inflammatory, patrolling monocyte activity (M. Gliem, et al. Biochem. Biophys. Acta. 1862, 329-338 (2016)) was more highly expressed in monocytes isolated from treated mice. Thus, treatment with sPD-L1 appears to shift the monocyte subpopulations from a Ly6hi, CCR2hi, CD43lo to a Ly6Clo, CX3CR1hi, CD43hi, PD-L1+ phenotype.

Given the prominence of metabolic pathways affected by sPD-L1 treatment and to confirm agonist activity of sPD-L1, the functional consequences of sPD-L1 and PD-1 blocking antibodies were studied in an in vitro myeloid precursor activation assay. This assay confirmed the in vivo observations. The results show that PD-1 blockade increased Ly6C expression at both low and high concentrations (p=0.0071 and 0.0157, respectively) while sPD-L1 supported low Ly6C expression. PD-1 blocking antibodies inhibited the effects of sPD-L1 in a dose-dependent manner (FIG. 5G). Using the same in vitro system, oxygen consumption rate (OCR) was measured and it was found that PD-1 blockade decreased OCR while sPD-L1 increased OCR (FIGS. 5H-I). sPD-L1 showed trends towards increased maximum (p=0.0793) and spare (0.0644) respiratory capacity, while anti-PD-1 blocked the effects of sPD-L1 at low doses and significantly decreased respiratory capacity from baseline at high doses. These data are consistent with the RNA sequencing data showing that sPD-L1 activates PD-1 during monocyte maturation, driving these cells to a restorative phenotype supported by specific metabolic programs.

Revascularization improves outcomes after LVO by limiting primary ischemic injury and is the cornerstone of treatment (F. A. Wollenweber, et al. Stroke. 50, 2500-2506 (2019); and F. Cagnazzo, et al., Journal of NeuroInterventional Surgery. 12, 350-355 (2020)); however, there are few options to mitigate secondary injury. Post-stroke inflammation plays a role in secondary injury, but the contributions of immune activation to cerebral edema, neuronal damage, and recovery are incompletely understood (J. Anrather and C. Iadecola, Neurotherapeutics. 13, 661-670 (2016); R. L. Jayaraj, et al., J Neuroinflammation. 16, 142 (2019); and K. Shi, et al. Lancet Neurol. 18, 1058-1066 (2019)). Several studies have focused on infiltrating monocytes and monocyte-derived macrophages (J. Yang, et al. Biomark Res. 2, 1 (2014); M. Kanazawa, et al. Int J Mol Sci. 18, 2135 (2017); and C. Tsou, et al. J Clin Invest. 117, 902-909 (2007)). However, these cells are difficult to study in situ because they undergo phenotypic changes upon entry into the brain (L. Garcia-Bonilla, et al., J Neuroinflammation. 13, 285 (2016); and J. Faustino, et al. J Cereb Blood Flow Metab. 39, 1919-1935 (2019)). Furthermore, subpopulations of monocytes and monocyte-derived macrophages play oppositional roles in recovery. For example, depleting monocytes or blocking their infiltration altogether is counterproductive, as inflammatory or M1-like (Ly6Chi) monocyte-derived macrophages transition to a regulatory or M2-like (Ly6Clo) phenotype to clear debris and promote healing (K. Rahman, et al. J Clin Invest. 127, 2904-2915 (2017); M. Kanazawa, et al. Int J Mol Sci. 18, 2135 (2017); and M. J. Crane, et al. PLOS One. 9, e86660 (2014)). An effective strategy, therefore, could be to reprogram monocytes in the blood before they migrate into the brain. The data described herein demonstrates that sPD-L1 acts on monocytes in the periphery to prevent harmful inflammation while promoting tissue repair programs after stroke.

Cerebral edema after stroke is multifactorial. Upregulation of the ion channel SURI is a driver of cerebral edema (J. M. Simard, et al., Nat Med. 12, 433-440 (2006)) and is the target of glyburide, which has been studied in clinical trials for patients with large hemispheric infarctions (Z. A. King, et al. Drug Des Devel Ther. 12, 2539-2552 (2018)). Immune activation contributes in part by increasing BBB permeability (Y. Huang, et al. Curr Neuropharmacol. 18, 1227-1236 (2020)), although targetable immune pathways have been elusive. In the patient cohort, it was found that PD-1 was expressed on blood monocytes and expression levels on intermediate and non-classical monocytes had the strongest correlations with cerebral edema. Human antibodies used for these experiments are shown in Table 10. Although larger patient cohorts are required to determine the potential of PD-1 expression as a biomarker, these data show that PD-1 is differentially expressed on monocyte subtypes after stroke. This finding is particularly intriguing as the strongest correlations were found with edema in the intermediate and non-classical subtypes. Intermediate monocytes have been associated with poor clinical outcomes in stroke patients and non-classical monocytes participate in tissue restoration (M. Kaito, et al. PLOS ONE. 8, c69409 (2013)). While there are no intermediate monocytes in rodents, it was found that sPD-L1 shifted monocytes from an inflammatory to a non-classical subtype. The human data are consistent with the notion that PD-1 activation on intermediate monocytes result in an analogous phenotypic switch.

In mice, sPD-L1 administration decreased brain water content by an average of 2.28% (0.251 mg) at 48 hours post-MCAO. These represent biologically significant changes, as previous studies have shown that modest changes in percent water content reflect substantial changes in rodent brain edema (27). MRIs obtained at 72 hours post-MCAO corroborated these data, as it was found that the volume of overall edema (T2 bright) normalized to the volume of infarct (ADC dark/DWI bright) was decreased with sPD-L1 administration. “Excess edema,” which was defined as the volume of hypointensity on ADC maps subtracted from the volume of T2 hyperintensity, was also measured to quantify the amount of tissue swelling that occurred outside of the core infarct. Given that differences in survival were driven primarily by deaths within the first 48 hours post-MCAO, it is likely sPD-L1 administration decreased intracranial pressure demonstrating that patients with large infarcts who are at risk of malignant cerebral edema are likely to derive the greatest benefit from peripheral PD-1 activation.

The current treatment for refractory malignant cerebral edema is decompressive hemicraniectomy, which is effective in preventing mortality after large territory infarction but fails to benefit functional recovery (J. Lin and J. A. Stroke. 52, 1500-1510 (2021); and K. Vahedi, et al., Stroke. 38, 2506-2517 (2007). Conversely, mechanical thrombectomy improves functional outcomes, but can exacerbate inflammation in the setting of reperfusion injury (F. C. Ng, et al. Stroke. 52, 3450-3458 (2021); and A. Mizuma, et al. Stroke. 49, 1796-1802 (2018)). These clinical observations show that functional outcomes are primarily driven by the extent of the ischemic injury, while inflammation plays a larger role in acute edema. Thus, as PD-L1 treatment did not reduce stroke volume, long-term functional measures were not significantly changed with sPD-L1 treatment. Subgroup analyses, however, demonstrated a role for PD-1-mediated inflammation in functional recovery.

The 28-point neuroscore is a well-established test to identify sensorimotor deficits (A. Encarnacion, et al. J Neurosci Methods. 196, 247-257 (2011)). While typically analyzed as a total composite score, it was found that these results were skewed by survival of more debilitated animals in the treatment group surviving the first 48-72 hours after MCAO. Therefore, a subgroup analysis was performed based on performance. sPD-L1 had minimal effect on mice below the 50th percentile. At week 1, however, there was a significant proportion of treated mice in the highest quartile that would have presumably been in the second quartile without treatment. These data showed that deficits due to severe ischemic injury cannot be overcome by limiting inflammation. Animals with more limited ischemic injuries, however, saw a functional benefit from sPD-L1 administration. After week 1, there were no differences in 28-point scores in either group, consistent with previous reports of early 28-point score recovery in rodent MCAO models (A. Encarnacion, et al., J Neurosci Methods. 196, 247-257 (2011)). If these findings translate to stroke patients, PD-1 agonists could expedite early mobilization and facilitate participation in physical rehabilitation.

Presuming that the neuroscore readouts were not sufficiently sophisticated to identify subtle long-term neurologic deficits, the DigiGait and open field tests were administered. Gait is a complex and interconnected outcome in rodents, as deficits in one limb can affect the other limbs in unpredictable ways (E. H. Lakes and K. D. Allen, Osteoarthritis Cartilage. 24, 1837-1849 (2016); and K. M. Chan, et al., 31, 425-434 (2023)). Several parameters of locomotor activity were identified that were significantly changed from baseline by MCAO and those parameters were evaluated at weeks 1 and 3. Although no single variable reached significance between the treated and untreated groups, most variables favored treatment, with 100% of the variables in favor of treatment at week 3. The same trend was found with the open field test administered 4 weeks after infarction as 80% of variables affected by stroke were improved with sPD-L1. Taken together, these data show that PD-1-mediated inflammation plays a modest, but measurable role in late outcomes.

There are conflicting data on the role of the PD-1/PD-L1 axis in stroke. Ren et al. proposed that PD-1 signaling is protective based on the finding that PD-1-deficient mice had larger infarct volumes and evidence of increased immune activation (X. Ren, et al., Stroke. 42, 2578-2583 (2011)). Although differences in infarct volumes were not observed, the data generated herein are consistent with these findings, as both global and myeloid-specific PD-1 knockout mice trended toward more edema than wild-type animals. The data further demonstrated that PD-1 signaling on monocytes skews towards a non-inflammatory program. Taken together, these data indicate that PD-1 is upregulated upon activation of inflammatory monocytes or their precursors and has an inhibitory effect when it binds its soluble ligands. Conversely, global knockout of PD-L1 and PD-L2 as well as PD-L1 blockade have been reported to decrease infarct volumes, reduce monocyte and CD4+ T cell infiltration, and improve neurologic outcomes (S. Bodhankar, et al., Stroke. 46, 2926-2934 (2015); and S. Bodhankar, et al., J Neuroinflammation. 10, 111 (2013)). It was also found that PD-1 is upregulated on infiltrating monocytes after ischemic stroke, and that the frequency of PD-1+ monocytes in the brain is decreased with systemic sPD-L1 administration. Of note, PD-1+ macrophage density in the brain was not different between the groups. Furthermore, PD-1 expression on monocytes in the blood was not different between the groups, likely due to transient PD-1 expression during phenotype switching, which is a well-known quality of monocytes (S. Canè, et al. Front Immunol. 10, 1786 (2019)). Thus, the tissue-specific knockout experiments that identify monocytes as the site of action of sPD-L1 afford an important insight into monocyte biology.

One explanation for the differential effects of PD-1 and PD-L1 ablation is that the context and location of PD-1/PD-L1 binding is important. Although the mechanisms of signaling downstream of PD-L1 are incompletely understood, evidence from cancer indicates that PD-L1 signals independently of PD-1 to promote cell survival (N. Patsoukis, et al. Sci Adv. 6, cabd2712 (2020)). It is plausible, therefore, that PD-L1 ablation or blockade reduces survival of inflammatory immune cells. The data support differential effects of PD-L1 based on context with the preponderance of data in favor of peripheral PD-1 activation. Similarly, soluble PD-L1 has context-dependent activity that determines its effects in health and disease. The data show that soluble PD-L1 may specifically affect maturation of myeloid cells by driving PD-1 signaling. Membrane-bound PD-L1 canonically interacts with PD-1 on lymphocytes in trans, which occurs concurrently with B7 molecules binding CD28 and peptide-loaded MHC molecules binding T cell receptors. Co-stimulatory signals are required for T cell inhibition as PD-1 activation blocks signaling downstream of the TCR and CD28 via SHP2 phosphatase activity (N. Patsoukis, et al. Sci Adv. 6, cabd2712 (2020)). In this context, sPD-L1 can block PD-1 ligating membrane-bound PD-L1 to paradoxically activate T cells (M. Song, et al. J Immunother. 34, 297-306 (2011)). Monocytes, however, are not subject to TCR and CD28 regulation and the data show that sPD-L1 acts as a PD-1 agonist in this context to reprogram these cells to a patrolling phenotype in vivo and in vitro. The data support sPD-L1 as a clinically viable PD-1 agonist for treating acute inflammation due to selective activity on PD-1 expressing myeloid cells.

In humans, circulating classical, pro-inflammatory CD14hi, CD16lo monocytes (which correspond to Ly6Chi, CD43lo in mice) are increased during the acute phase of stroke, while the proportion of non-classical, anti-inflammatory CD14lo, CD16hi monocytes (Ly6Clo, CD43hi in mice) are decreased (M. Kaito, et al., PLOS One. 8, e69409 (2013)). The single-cell transcriptomic analysis of circulating monocytes similarly identified two populations of CD11b+ monocytes that differentially expressed Ly6C, CD43, and CCR2. Cell clusters predominantly populated by untreated monocytes displayed stronger expression of Ly6C and CCR2 and weaker expression of CD43 (consistent with a classical phenotype), whereas clusters predominantly populated by monocytes from treated mice displayed stronger expression of CD43 and weaker expression of Ly6C and CCR2 (consistent with a non-classical phenotype). Of note, CCR2 expression levels were slightly higher with treatment across clusters. This heterogeneity is consistent with previous literature on CCR2 expression and outcomes after stroke as retention of CCR2 expression on monocyte precursors is important for post-stroke recovery while CCR2 on mature monocytes does not have the same effect (J. Pedragosa, et al., J. Cereb. Blood Flow Metab. 40, S98-S116 (2020)). PD-L1 treated monocytes also displayed significant negative enrichment for inflammatory and defense pathways, and positive enrichment for metabolic pathways. Furthermore, the data show that PD-1 blockade decreases oxidative phosphorylation in opposition to sPD-L1 in vitro. As increased oxidative phosphorylation is a hallmark of M2 macrophage polarization these data confirm functional effects of sPD-L1 on monocyte maturation to a restorative phenotype (A. Viola, et al. Front Immunol. 10, 1462 (2019)) and further support sPD-L1 acting as a PD-1 agonist in opposition to PD-1 blocking antibodies.

It is important to note that the effects of PD-1 signaling on monocytes appears to be disease-dependent as there is evidence that PD-1 blockade augments the influx of monocytes into the brain in Alzheimer's disease (AD) models (N. Rosenzweig, et al. Nat Commun. 10, 465 (2019); K. Baruch, et al. Nat Med. 22, 135-137 (2016); and M. Ghareghani and S. Rivest, Int J Mol Sci. 24, 10905 (2023)). In this context, intraperitoneal injection of anti-PD-L1 antibody increased trafficking of Ly6Chi CD45hiCD11b+ cells from the periphery into the brain (N. Rosenzweig, et al. Nat Commun. 10, 465 (2019); and K. Baruch, et al. Nat Med. 22, 135-137 (2016)) mediated by CCR2 signaling (H. Ben-Yehuda, et al. Mol Neurodegener. 16, 39 (2021)). In contrast, it was found that PD-1 activation with sPD-L1 had modest impact on monocyte recruitment, but instead initiated a subset switch in the periphery after stroke. Taken together these findings highlight the importance of context when considering targets in acute inflammation versus chronic or degenerative disease states. With the former, the therapeutic benefit may lie in “defusing” the early and massive influx of proinflammatory monocytes; with the latter, the incoming Ly6C+ monocyte-derived macrophages likely arrive over a longer period of time, and comprise various activation states with disease-modification functions (N. Rosenzweig, et al. Nat Commun. 10, 465 (2019)), thus there may be a greater benefit to facilitating their migration into the CNS.

The Ly6Chi, CD43lo, CCR2+ phenotype has been previously described as a proinflammatory subset of monocytes that is recruited early to the ischemic brain tissue, while the nonclassical Ly6Clo, CD43hi, CX3CR1+ phenotype is less responsive to inflammatory stimuli (F. Miró-Mur, et al. Brain Behav Immun. 53, 18-33 (2016). These blood-derived monocytes and macrophages (Mo/Mf) have been shown to accumulate sequentially in the ischemic brain (L. Garcia-Bonilla, et al. J Neuroinflammation. 13, 285 (2016)), with an influx of Ly6Chi Mo/Mf into the infarct core 3-5 days post-MCAO, and perivascular Ly6Clo Mo/Mf in the surrounding penumbra. Importantly, the accumulation of Ly6Clo monocytes in the surrounding brain is due to a phenotypic switch (or further maturation) of Ly6Chi monocytes, rather than recruitment of a separate monocyte population (L. Garcia-Bonilla, et al. J Neuroinflammation. 13, 285 (2016); and F. Miró-Mur, et al. Brain Behav Immun. 53, 18-33 (2016)).

The data indicate that PD-1/PD-L1 signaling plays a role in this maturation process. Strauss et al. previously reported that myeloid-specific PD-1 ablation caused increased accumulation of Ly6Chi monocytes in subcutaneous flank tumors (L. Strauss, et al. Sci Immunol. 5, eaay 1863 (2020)). More recently, the same group demonstrated that myeloid-specific knockout of the tyrosine phosphatase SHP-2 inhibited maturation and resulted in increased infiltration of Ly6Chi monocytes into subcutaneous tumors (A. Christofides, et al. Nat Immunol. 24, 55-68 (2023)). Thus, both PD-1 and SHP-2 signaling appear to have important roles in myeloid lineage fate commitment (L. Garcia-Bonilla, et al. J Neuroinflammation. 13, 285 (2016); and F. Miró-Mur, et al. Brain Behav Immun. 53, 18-33 (2016)). When the differentially expressed genes from our wild-type mice were compared against those published from SHP-2 knockout mice, negative enrichment for over-expressed genes after SHP-2 knockout (p=1.149e-24), and positive enrichment for under expressed genes (p=3.725e-45) was found. These results indicate activation of canonical downstream pathways with PD-L1 administration. A separate differential expression analysis of treated versus non-treated monocytes using M1 and M2 macrophage markers curated from the literature (F. Szulzewsky, et al., PLOS One. 10, e0116644 (2015)) showed significant negative enrichment of M1 markers (p=0.0016) (FIG. 10). Though it should be noted that not all were monocyte-specific markers, the skew away from M1-associated markers may indicate an M2-type lineage commitment for PD-1 activated monocytes. Taken together, these data show that PD-1 binding to PD-L1 switches monocyte fates away from pro-inflammatory or classical monocytes, and toward non-classical, anti-inflammatory phenotypes prior to tissue infiltration. The long-term functional outcomes in these studies show this it is likely that Ly6Clo monocytes in the circulation after PD-L1 treatment eventually traffic to the brain to serve a supportive role, and demonstrate that PD-1/PD-L1 binding on blood monocytes can improve early outcomes without compromising long-term recovery, making peripheral PD-1 activation a therapeutic approach that can be applied to limit inflammatory injury after large territory stroke.

TABLE 10
Antibodies used for flow cytometry of human blood samples

| Antibody (Flow) | Clone | Fluorophore | Dilution | Company |
|---|---|---|---|---|
| CD45 | HI30 | AF700 | 1:100 | Biolegend |
| CD11b | ICRF44 | BV421 | 1:100 | Biolegend |
| CD14 | HCD14 | APC-Cy7 | 1:50 | Biolegend |
| CD16 | 3G8 | PE-Cy7 | 1:50 | Biolegend |
| PD-1 | EH12.2H7 | PE | 1:100 | Biolegend |
| CD3 | SK7 | PercpCy5.5 | 1:20 | Biolegend |
| CD15 | H198 | FITC | 1:50 | Invitrogen |
| CD19 | HIB19 | FITC | 1:100 | Biolegend |
| CD11c | 3.9 | BV650 | 1:100 | Biolegend |
| CD8 | Sk1 | BV605 | 1:100 | BD Biosciences |
| Tim-3 | F38-2E2 | APC | 1:100 | Biolegend |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of reducing or preventing cerebral edema or decreasing intracranial pressure in a subject after a stroke, the method comprising: administering systemically a therapeutically effective amount of a PD-1 agonist to the subject.

2. (canceled)

3. A method of improving gait or sensorimotor deficits in a subject after a stroke, the method comprising: administering systemically a therapeutically effective amount of a PD-1 agonist to the subject.

4.-6. (canceled)

7. The method of claim 1, wherein the intracranial pressure is reduced by at least 1%.

8. A method of shifting the phenotype of monocytes in the brain from a classical inflammatory subtype to a non-classical subtype in a subject after a stroke, the method comprising: administering systemically a therapeutically effective amount of a PD-1 agonist to the subject.

9. The method of claim 8, wherein the classical inflammatory subtype is a CD14^{hi}, CCR2^{hi}, CD16^{lo} phenotype.

10. The method of claim 8, wherein the non-classical subtype is a CD14^{lo}, CX3CR1^{hi}, CD16^{hi}, PD-L1+ phenotype.

11. (canceled)

12. (canceled)

13. The method of claim 1, wherein the subject had an acute ischemic stroke or a subarachnoid hemorrhage.

14. The method of claim 13, wherein the acute ischemic stroke was caused by a large vessel occlusion.

15. The method of claim 1, wherein the PD-1 agonist is a soluble PD-L1 or an analogue thereof.

16. The method of claim 15, wherein the soluble PD-L1 or the analogue thereof is a PD-L1 fusion protein.

17. The method of claim 16, wherein the PD-L1 fusion protein is SEQ ID NO: 12 or SEQ ID NO: 13.

18. The method of claim 1, wherein the PD-1 agonist is an antibody or antigen-binding fragment thereof.

19. The method of claim 1, wherein the systemic administration is intravenous, intraperitoneal, or oral.

20. The method of claim 1, wherein the PD-L1 agonist is administered between 1 s and 72 hours after the subject had the stroke.

21. The method of claim 1, wherein after the stroke, in the acute period, PD-1 expression on monocytes is upregulated in the brain.

22. The method of claim 21, wherein the administration of the PD-L1 agonist activates PD-1 on peripheral monocytes, and wherein the activated peripheral monocytes infiltrate the brain, thereby decreasing the number of PD-1 positive monocytes in the brain.

23. The method of claim 1, wherein the subject is a human.
